(19) United States
(12) Patent Application Publication
(10) Pub. No.: US 2026/0167585 A1
(43) Pub. Date: Jun. 18, 2026

(54) DEUTERATED COMPOSITION, PREPARATION METHOD THEREFOR, AND ORGANIC ELECTROLUMINESCENT DEVICE COMPRISING DEUTERATED COMPOSITION

(71) Applicant: SHIJIAZHUANG CHENGZHI YONGHUA DISPLAY MATERIAL CO., LTD., Shijiazhuang (CN)

(72) Inventors: Tianyu WEI, Shijiazhuang (CN); Feng LIANG, Shijiazhuang (CN); Jing ZHANG, Shijiazhuang (CN); Lingwei XU, Shijiazhuang (CN); Zhicheng CAO, Shijiazhuang (CN)

(73) Assignee: SHIJIAZHUANG CHENGZHI YONGHUA DISPLAY MATERIAL CO., LTD., Shijiazhuang (CN)

(21) Appl. No.: 19/530,010

(22) Filed: Feb. 4, 2026

(30) Foreign Application Priority Data
Aug. 21, 2025 (CN) 202511178617.7

(51) Int. Cl. C07B 59/00; C07D 307/87; H10K 50/11; H10K 85/60

(52) U.S. Cl. CPC C07B 59/002; C07D 307/87; H10K 50/11; H10K 85/626; H10K 85/631; H10K 85/654; H10K 85/6572; H10K 85/6574; H10K 85/658; C07B 2200/05

(57) ABSTRACT

The present invention discloses a deuterated composition, a preparation method therefor, and an organic electroluminescent device comprising the deuterated composition. The deuterated composition comprises at least two compounds represented by Formula I or at least two compounds represented by Formula II. By means of the deuterated composition composed of the compounds having specific deuteration positions and degrees of deuteration, the prepared organic electroluminescent device can improve a crystallization problem caused by a decrease in the uniformity of a thin film of a luminescent layer caused by frequent temperature changes while the voltage of the device is maintained basically unchanged, and thus improves the service lifetime of the organic electroluminescent device in a complex temperature environment. Furthermore, the present invention further provides a method for preparing the deuterated composition. The preparation method can precisely control the deuteration position and the degree of deuteration of the compound, thereby facilitating obtaining the desired deuterated composition.

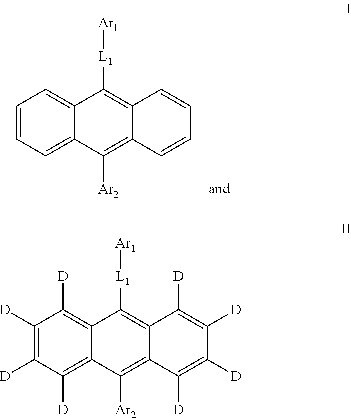

### TECHNICAL FIELD

The present invention relates to the technical field of OLEDs. More specifically, the present invention relates to a deuterated composition, a preparation method therefor, and an organic electroluminescent device comprising the deuterated composition.

### BACKGROUND ART

Organic Light-Emitting Diodes (OLEDs) are a very popular display technology. Due to the innovation of the structure of organic electroluminescent devices and the use of efficient transmission materials, host materials, luminescent materials, and injection materials, display and lighting applications based on the OLED technology have become currently one of the most competitive technologies. Due to the characteristics of high efficiency, lightweight, high color display quality, near 1800 viewing angles, ultra-fast response, the potential to achieve pure black display, etc., the OLED technology has attracted worldwide attention.

During the preparation of organic electroluminescent devices and the use of organic electroluminescent display devices, frequent temperature changes may occur. Such temperature changes may lead to a decrease in the stability of thin films of organic electroluminescent device, resulting in poor display issues. It has been found from research that for blue-light host materials, which are high-purity compounds composed of large conjugated segments, frequent temperature changes can more easily induce a material crystallization problem, thereby leading to a reduction in device lifetime.

In organic electroluminescent devices, deuterated materials have been widely used in various functional layers, especially in a luminescent layer. Deuterated materials achieve “passivation” due to reduced activity, thereby improving device lifetime. Due to the significantly lower vibrational energy of C-D (carbon-deuterium) bonds than C—H (carbon-hydrogen) bonds, this more stable vibrational mode enhances an intermolecular stacking effect; however, the enhancement of the intermolecular stacking effect exacerbates the material crystallization problem. In addition, an excessively strong stacking effect can also cause spectral redshift and energy loss.

In addition, current preparation methods for deuterated materials usually use traditional acid-catalyzed reactions of displacement of hydrogen with deuterium to complete deuterium substitution. This method has the problems of high pollution, large usage of deuterated water, difficulty in product purification, etc. In addition, due to the high activity of strong acids, it is difficult to control the deuteration percentage and deuteration position, and relatively strong reaction conditions are needed to directly prepare fully deuterated products. This may lead to substrate decomposition, resulting in low product yield and poor lifetime issues.

Therefore, in view of the above existing issues, it is very important to develop a deuterated blue-light host material more excellent in performance and a preparation method therefor.

### SUMMARY OF THE INVENTION

In view of the above problems existing in the prior art, the present invention provides a deuterated composition, a preparation method therefor, and an organic luminescent device comprising the deuterated composition.

In order to achieve the above objective, the following technical solution is used in the present invention:

In a first aspect, the present invention provides a deuterated composition, comprising at least two compounds represented by Formula I or at least two compounds represented by Formula II:

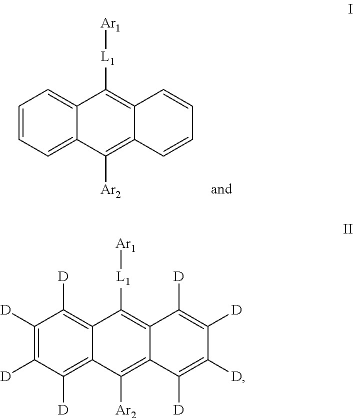

wherein
L₁ represents a single bond or phenylene;
Ar represents deuterated or undeuterated phenyl or naphthyl;
Ar₂ represents one of the following structures:

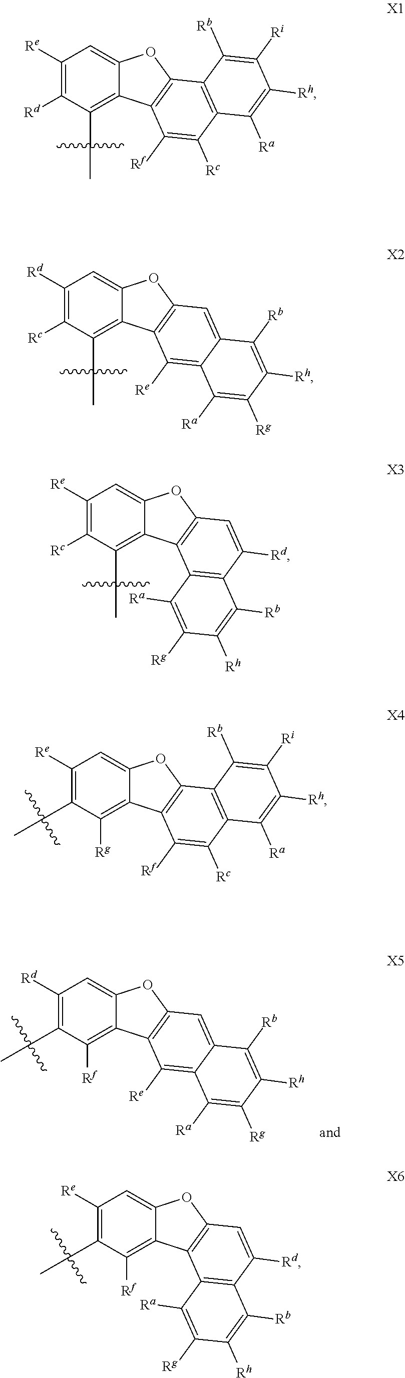

R^{a}, R^{b}, R^{c}, R^{d}, R^{e}, R^{f}, R^{g}, R^{h}, and Rⁱ are the same or different, each independently represent H or D, and satisfy the following conditions:
1) at least one of R^{a} and R^{b} is D; and
2) the degree of deuteration of Ar₂ is 10-60%, preferably, the degree of deuteration of Ar₂ is 20-40%, and more preferably, the degree of deuteration of Ar₂ is 20-30%.

Furthermore, at least one of R^{c}, R^{d}, and R^{e} is D; and/or at least one of R^{f}, R^{g}, R^{h}, and Rⁱ is D.

Furthermore, R^{a} and R^{b} are both D, and R^{c}, R^{d}, R^{e}, R^{f}, R^{g}, R^{h}, and Rⁱ are H or D.

Furthermore, R^{a}, R^{b}, and R^{c} are all D, and R^{c}, R^{d}, R^{f}, R^{g}, R^{h}, and Rⁱ are H or D.

Furthermore, R^{a}, R^{b}, R^{c}, and R^{d} are all D, and R^{c}, R^{f}, R^{g}, R^{h}, and Rⁱ are H or D.

Furthermore, R^{a}, R^{b}, R^{c}, R^{d}, and R^{c} are all D, and R^{f}, R^{g}, R^{h}, and Rⁱ are H or D.

Furthermore, Ar₁ represents one of the following structures:

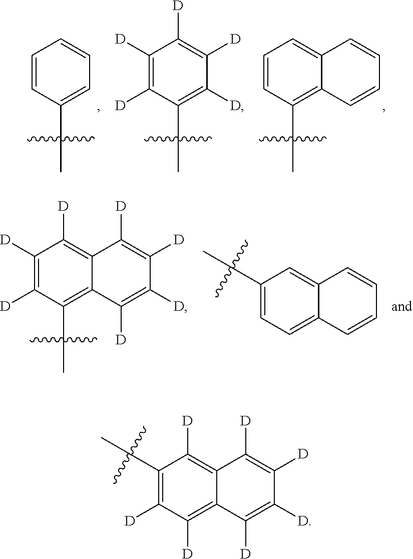

Furthermore, in X1 and X4, the degrees of deuteration of R^{a} and R^{b} are both 60%, the degrees of deuteration of R^{c}, R^{d}, and R^{c} are all more than 30%, the degrees of deuteration of R^{f} and R^{g} are both less than 20%, and the degrees of deuteration of R^{h} and Rⁱ are both less than 10%.

Furthermore, in X2 and X5, the degrees of deuteration of R^{a} and R^{b} are both more than 60%, the degrees of deuteration of R^{c} and R^{d} are both more than 30%, the degrees of deuteration of R^{e} and R^{f} are both less than 20%, and the degrees of deuteration of R^{g} and R^{h} are both less than 10%.

Furthermore, in X3 and X6, the degrees of deuteration of R^{a} and R^{b} are both more than 60%, the degrees of deuteration of R^{c}, R^{d}, and R^{c} are all more than 30%, the degree of deuteration of R^{f} is less than 20%, and the degrees of deuteration of R^{g} and R^{h} are both less than 10%.

Furthermore, in X1 to X6, the degree of deuteration of R^{a} or R^{b} is more than the degree of deuteration of R^{c}, R^{d}, or R^{e}, and the degree of deuteration of R^{c}, R^{d}, or R^{e} is more than the degree of deuteration of R^{f}, R^{g}, R^{h}, or Rⁱ.

It needs to be noted that the present invention achieves a highly selective deuteration reaction at an active site of oxa-aryl by using a highly selective metal catalyst, and the catalyst described in the present invention is used for the deuteration reaction without causing deuteration on a non-aromatic hydrocarbon, thereby improving the utilization efficiency of deuterium atoms. By using deuterated water as a deuterium source, a high degree of deuteration at a specific site is achieved while the overall degree of deuteration is maintained at a relatively low level. The amount of deuterium atoms used is reduced, the atomic economy is improved, the cost is significantly lowered, and the device performance after high-temperature processing is significantly improved.

Furthermore, Formula I is selected from one of the following structures:

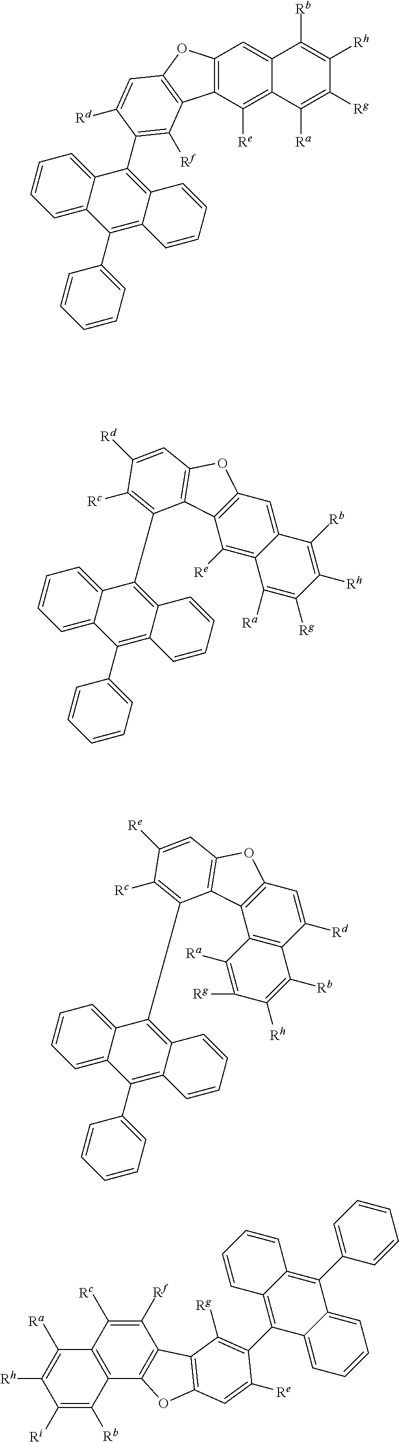

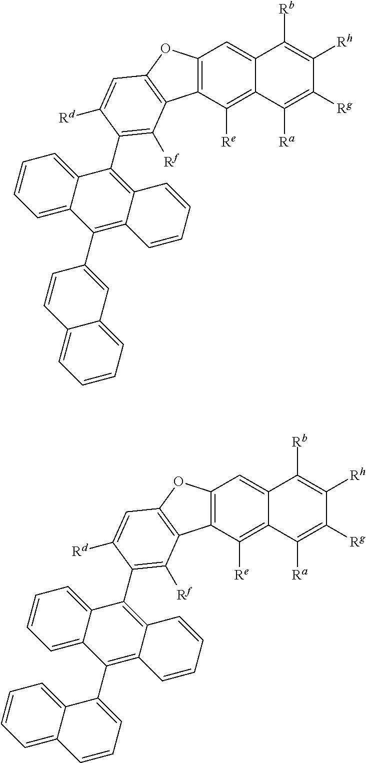

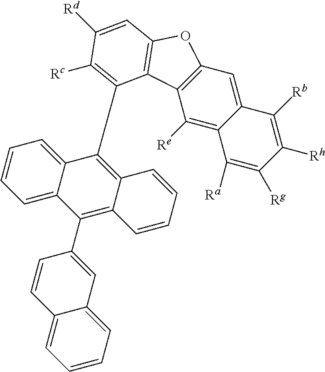

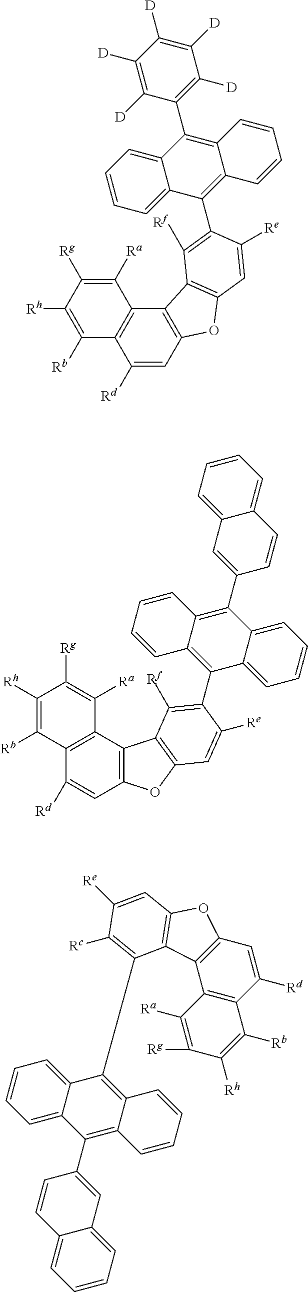

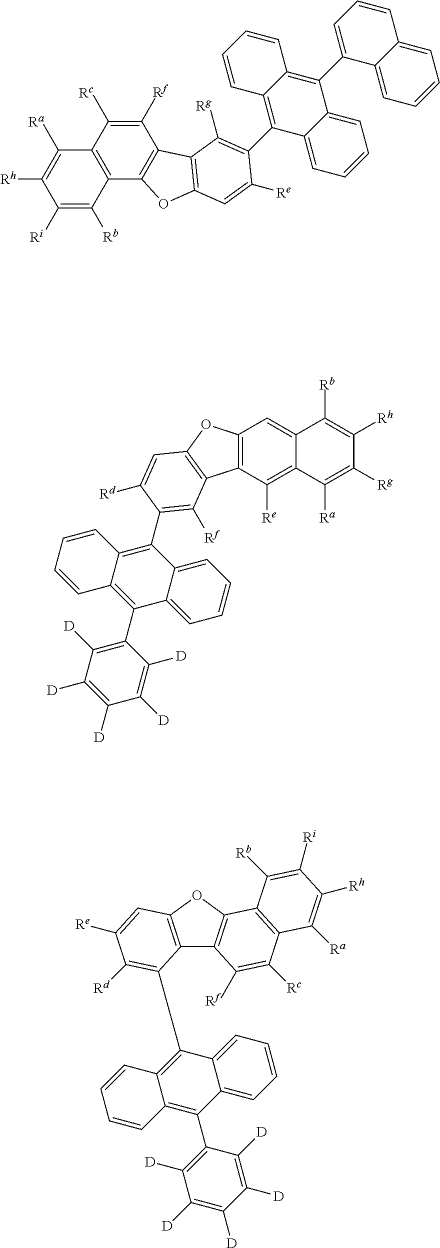

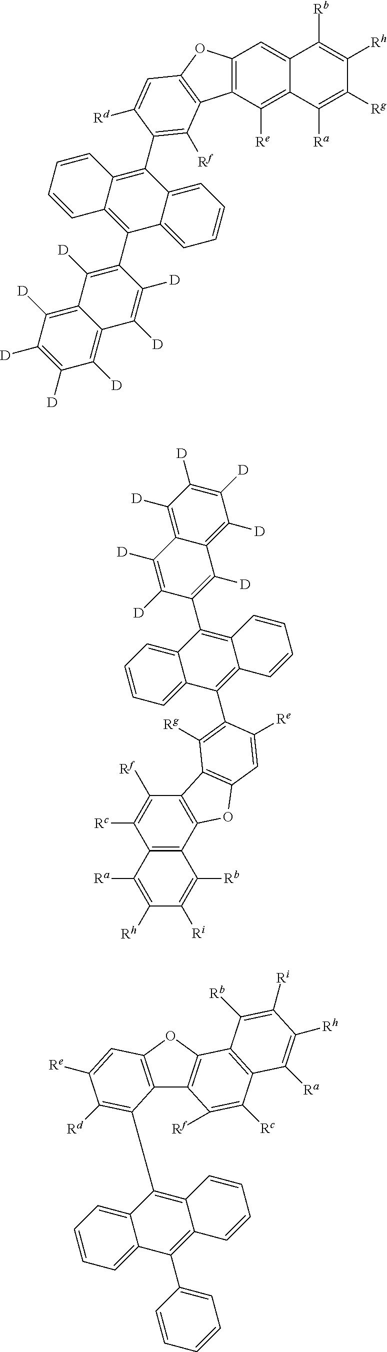

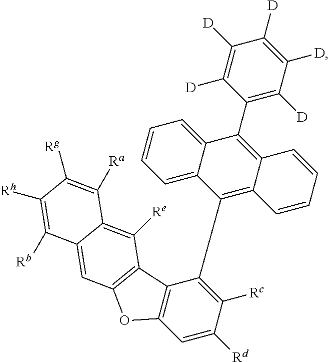

and

Formula II is selected from one of the following structures:

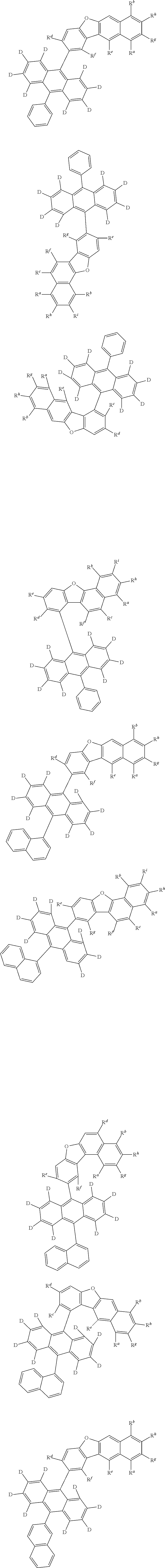

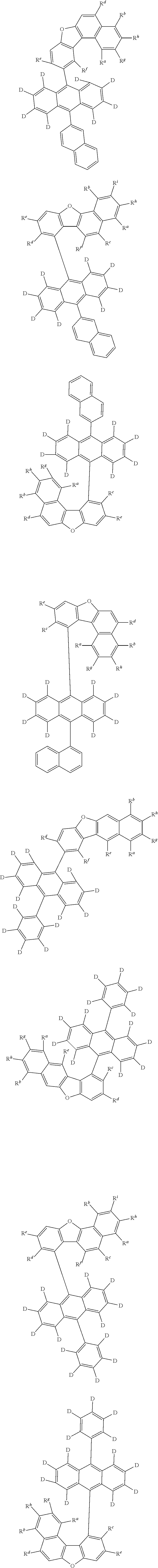

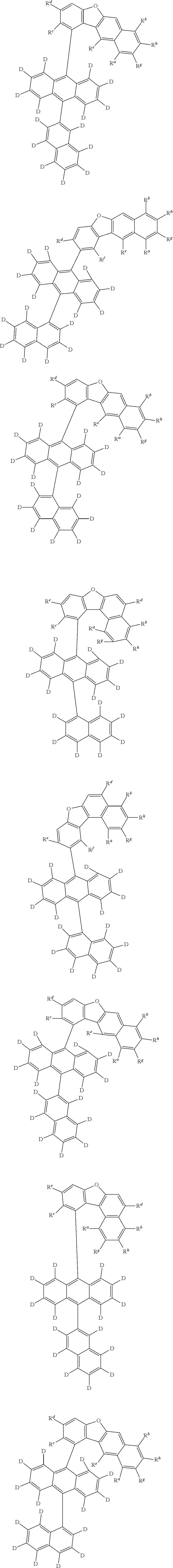

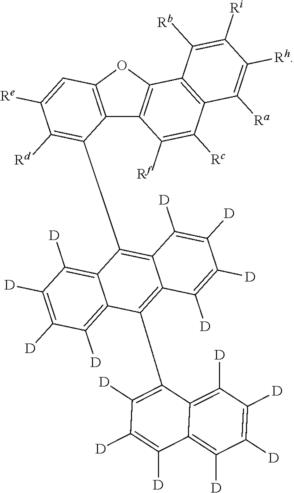

In a second aspect, the present invention provides a method for preparing the deuterated composition as described above, comprising the following steps:

S1. adding raw material I, deuterated water, an activated supported catalyst, and an organic solvent to a reaction vessel, and heating and then reacting the mixture to obtain a deuterated composition intermediate, the raw material I being selected from one of structures represented by the following formulas Sub-1 to Sub-6:

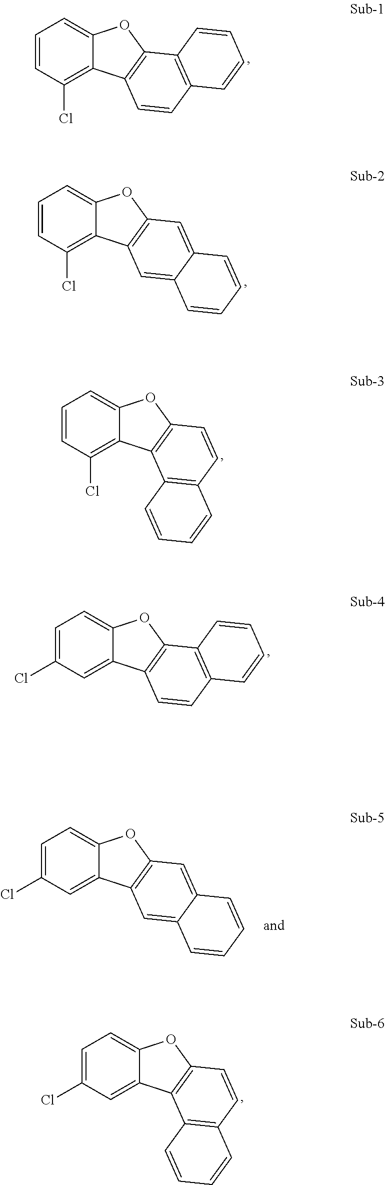 [SMILES: Clc1cccc2oc3c4ccccc4ccc3c12 ; Clc1cccc2oc3cc4ccccc4cc3c12 ; Clc1cccc2oc3ccc4ccccc4c3c12 ; Clc1ccc2oc3c4ccccc4ccc3c2c1 ; Clc1ccc2oc3cc4ccccc4cc3c2c1 ; Clc1ccc2oc3ccc4ccccc4c3c2c1]

wherein the mass ratio of raw material I to deuterated water is 5:1 to 100:1; and

Furthermore, the reaction temperature in step S1 is 80° C. or higher, and the reaction time is 1-24 h.

Furthermore, the volume ratio of the organic solvent to the deuterated water is 1:10 to 10:1.

Furthermore, the organic solvent is selected from oxacycloalkyl groups, such as tetrahydrofuran, methyltetrahydrofuran, or tetrahydropyran.

It has been found from research that the deuterated composition intermediate obtained by this preparation method is typically a mixture containing compounds having various deuteration patterns. Those skilled in the art can further analyze the degree of deuteration at each specific site of deuteration within the deuterated mixture intermediate in conjunction with proton nuclear magnetic resonance hydrogen spectroscopy is technology. Those skilled in the art has surprisingly found that the compound prepared by the method of the present invention exhibited a significantly improved degree of deuteration at the active site thereof, which is significantly different from the degree of deuteration at a non-active site.

S2. subjecting raw material II, potassium carbonate, a palladium catalyst, and a deuterated composition intermediate to a coupling reaction to obtain the deuterated composition, the raw material II being selected from one of structures represented by the following formulas A-1 to A-2:

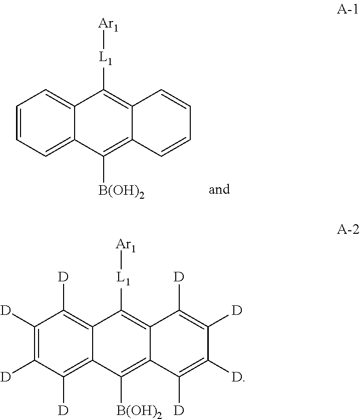

Furthermore, the molar ratio of the raw material II to potassium carbonate is 1:1 to 1:5.

Furthermore, the molar ratio of the raw material II to the palladium catalyst is 1:0.005 to 1:0.1.

Furthermore, the molar ratio of the raw material II to the deuterated composition intermediate is 0.9:1 to 1:0.9.

The coupling reaction in step S2 is a relatively mature process. It has been found from research that by screening coupling reaction conditions, the degree of deuteration at each site of deuteration in the original deuterated composition intermediate will not be destroyed when the raw material II and the deuterated composition intermediate are coupled. Therefore, when calculating the degree of deuteration at each site of deuteration in the deuterated composition, those skilled in the art can actually perform statistical analysis by means of the degree of deuteration of the deuterated composition intermediate.

In the preparation method provided by the present invention for the deuterated composition, selective deuteration at an active site of oxa-aryl by using a catalyst loaded with an active metal affords a range of composition intermediates with determined deuteration positions, and the composition intermediates are then subjected to a coupling reaction with an arylphenylboronic acid raw material to obtain a range of aromatic compositions having determined deuteration positions, so that the deuterated compositions having precisely controlled deuteration positions and deuteration percentages are realized.

Furthermore, the reaction temperature of the coupling reaction is 80-150° C., and the reaction time is 4-24 h.

Furthermore, the supported catalyst is prepared by the following steps:
adding a platinum salt and a palladium salt to dilute hydrochloric acid, stirring and dissolving the platinum salt and palladium salt, then adding a porous material, and subjecting the mixture to impregnation, filtration, drying, and calcination to obtain the supported catalyst,
wherein based on the metal mass, the mass ratio of the porous material to the platinum salt and the palladium salt is 10:0.1 to 10:5.

Furthermore, the platinum salt is selected from one or more of platinum tetrachloride, platinum dichloride, platinum nitrate, and chloroplatinic acid.

Furthermore, the palladium salt is selected from one or more of palladium chloride, palladium nitrate, palladium acetate, or dichlorotetraamminepalladium.

Furthermore, the porous material is selected from one of activated carbon, a is molecular sieve, activated aluminum oxide, and silica gel, preferably a molecular sieve, such as ZSM-5.

Furthermore, the impregnation time is 24 hours or more.

Furthermore, the drying is vacuum drying or forced-air drying, the drying temperature is 50° C. or less, and the drying time is 2 hours or more.

Furthermore, the calcination temperature is 150° C. or higher, and the calcination time is 2 hours or more.

In a third aspect, the present invention provides an organic electroluminescent device, comprising an anode, a hole transport region, a luminescent layer, an electron transport region, and a cathode, which are sequentially arranged on a substrate plate, wherein the luminescent layer comprises a host material and a guest material, and the host material comprises the deuterated composition as described above.

### Beneficial Effects of the Invention

The present invention provides a deuterated composition, which comprises two or more compounds for which the deuteration position and the degree of deuteration are precisely controlled. By combining the above materials, the degree of deuteration of the deuterated composition at a specific site of deuteration can be further controlled, so that the prepared organic electroluminescent device can improve the crystallization problem caused by a decrease in the uniformity of the thin film caused by temperature changes while the voltage of the device is maintained basically unchanged, and thus improves the service lifetime of the organic electroluminescent device in a complex temperature environment.

The present invention further provides a method for preparing the deuterated composition. The method realizes a process of the high-activity and high-selectivity displacement of hydrogen with deuterium by using a composite catalyst loaded with an active metal, thus preparing a special deuterated composition. The preparation method provided by the present invention for the deuterated composition can precise control the is deuteration position and deuteration percentage in the compound, which is more beneficial to controlling the degree of deuteration at a specific site of deuteration in the deuterated composition, laying the groundwork for the final preparation of an organic electroluminescent device having excellent performance. The method has high stability, and the finally obtained target product has relatively high yield.

The organic electroluminescent device provided by the present invention has relatively driving voltage, relatively high luminous efficiency, and relatively long service lifetime.

### BRIEF DESCRIPTION OF THE DRAWINGS

FIG. 1 is a schematic structural diagram of an organic electroluminescent device of the present invention, wherein 1—substrate, 2—anode, 3—hole injection layer, 4—hole transport layer, 5—luminescent auxiliary layer, 6—luminescent layer, 7—hole barrier layer, 8—electron transport layer, 9—electron injection layer, 10—cathode, and 11—cover layer.

### DETAILED DESCRIPTION OF EMBODIMENTS

In order to understand the content of the present invention more clearly, the present invention will be described in detail in conjunction with the accompanying drawings and examples.

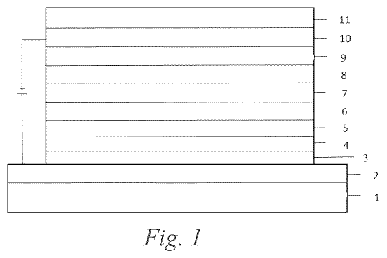

The deuterated composition of the present invention is suitable for light-emitting elements, display panels, and electronic devices, especially suitable for organic electroluminescent devices. The electronic device of the present invention is a device comprising a layer of at least one organic compound, and the device may also comprise an inorganic material or a layer formed entirely of an inorganic material. The electronic device is preferably an organic electroluminescent device (OLED), an organic integrated circuit (0-IC), an organic field effect transistor (0-FET), an organic thin film transistor (0-TFT), an organic light-emitting transistor (0-LET), an organic solar cell (0-SC), an organic dye-sensitized solar cell (0-DSSC), an organic optical detector, an organic photosensor, an organic field-quenching device (0-FQD), a luminescent electrochemical cell (LEC), an organic laser diode (0-laser), and an organic plasma emitting device. The electronic device is preferably an organic electroluminescent device (OLED). The schematic structural diagram of an exemplary organic electroluminescent device is as shown in FIG. 1.

### Synthesis of Deuterated Compositions

### Synthesis Example 1

### Step 1: Impregnation of Catalyst Carrier

To a beaker was added 100 ml of dilute hydrochloric acid having a concentration of 5% (w.t.). 3.4 g of platinum tetrachloride and 1.8 g of palladium chloride were then added to the dilute hydrochloric acid and stirred at room temperature for dissolution. 10 g of ZSM-5 molecular sieve was then added to the beaker and soaked for 12 hours.

### Step 2: Calcination and Shaping of Catalyst

The molecular sieve was separated from the dilute hydrochloric acid solution using a filter cloth and washed three times with water to remove excess hydrochloric acid. The molecular sieve loaded with platinum and palladium was then placed in a blast drying oven and dried at 120° C. for 4 h to remove moisture on the surface and adsorbed moisture. Finally, the molecular sieve was then calcined at 200° C. for 2 hours to obtain a catalyst, denoted as ZSM-5-Pt-Pd-1.

### Step 3: Activation of Catalyst

The prepared catalyst ZSM-5-Pt-Pd-1 was added again to the reactor, followed by the addition of 250 ml of water to the reaction system. The mixture was then stirred at room temperature under 3 atm hydrogen pressure for 4 hours. Subsequently, excess water was decanted, and the remaining catalyst was dried in vacuo under the drying conditions of 40° C., 2 hours, and a vacuum degree below 10 torr to obtain an activated catalyst, denoted as ZSM-5-Pt-Pd-1H.

### Step 4: Preparation of Deuterated Composition Intermediate

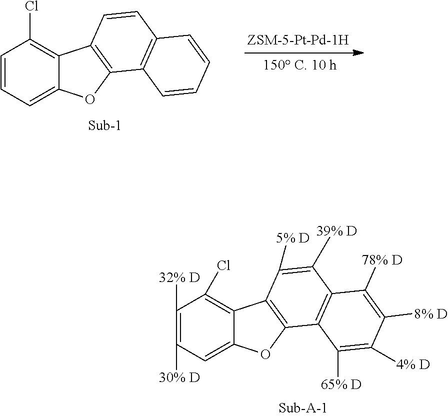

To a 500 ml autoclave were added 200 ml of deuterated water, 50 ml of tetrahydrofuran, and 3 g of the activated catalyst ZSM-5-Pt-Pd-1H, followed by the addition of the raw material represented by Sub-1 (20 g, 79 mmol). The mixture was then stirred at 150° C. for 10 hours. The reaction system was then cooled to room temperature and filtered to remove excess solvent. The filter cake was dissolved in 200 ml of toluene. After suction filtration, the filtrate was concentrated and recrystallized three times using a mixed liquid of dichloromethane and n-hexane (the volume ratio of dichloromethane to n-hexane being 1:1) to obtain the deuterated composition intermediate Sub-A-1 (15.2 g).

¹H NMR (400 MHz) δ 8.50-8.48 (0.35H), 8.46-8.44 (0.92), 8.06-8.04 (0.96H), 7.87-7.85 (0.22H), 7.72-7.70 (0.61), 7.69-7.66 (1H), 7.64-7.63 (0.61H), 7.46-7.44 (0.68H), 7.42-7.41 (0.70H).

After comprehensive analysis by nuclear magnetic resonance and mass spectrometry, the average degree of deuteration of the deuterated composition intermediate was determined to be 29% (i.e., the degree of deuteration of the Ar₂ group was 29%). The statistical data of the degree of deuteration at each site of deuteration in the structure of the deuterated composition intermediate was as shown in the reaction equation.

In addition, the average degree of deuteration of the deuterated composition intermediate was calculated by the following method:
average degree of deuteration=(a %+b %+c %+d %+e %+f %+g %+h %+i %)/(n−1), in which a % to i % are the degrees of deuteration at positions R^{a} to Rⁱ, respectively; n is the number of sites in the deuterated composition intermediate that can be substituted by deuterium or other groups (e.g., Cl); and in Sub-A-1, n is 10.

### Step 5: Preparation of Deuterated Composition

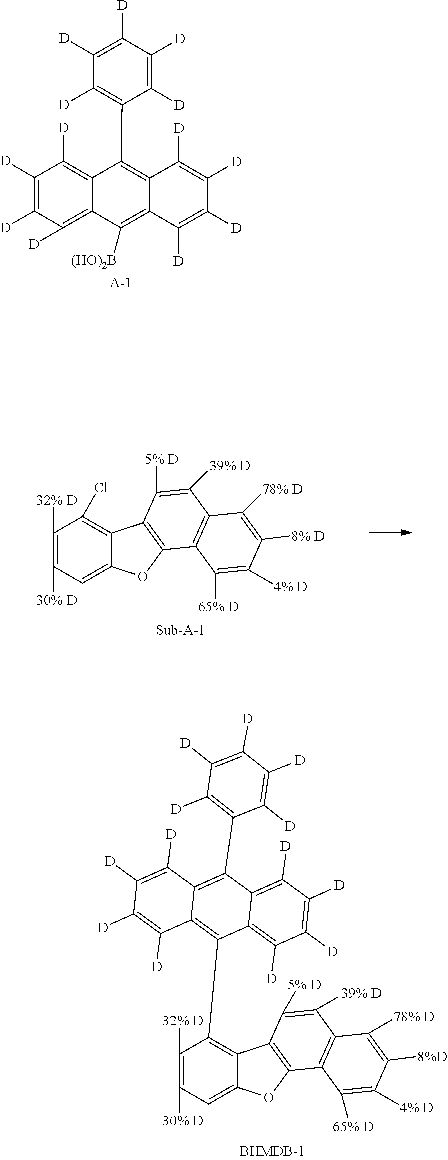

To a 250 ml three-necked flask were added a raw material represented by Formula A-1 (3.1 g, 10 mmol), deuterated composition intermediate Sub-A-1 (2.8 g), potassium carbonate (2.1 g, 15 mmol), tetrakis(triphenylphosphine)palladium (0.58 g, 0.5 mmol), and 100 ml of DMF. The mixture was uniformly stirred under nitrogen protection, then heated to 100° C. under nitrogen protection, and reacted for 4 hours. The reaction system was then cooled to room temperature, and 500 ml of toluene and 500 ml of water were added for liquid separation. The organic phase was concentrated and then purified using a column to obtain 2.0 g of deuterated composition BHMDB-1, with a molecular weight of 484-489.

¹H NMR (400 MHz) δ 8.54-8.52 (0.35H), 7.99-7.97 (1H), 7.77-7.74 (0.92H), 7.72-7.70 (0.96H), 7.66-7.64 (0.61H), 7.55-7.53 (0.68H), 7.53-7.51 (0.61H), 7.23-7.21 (0.22H), 6.19-6.16 (0.70H).

As can be seen from the nuclear magnetic resonance test results, the degree of deuteration at each site in the product was consistent with that in the deuterated composition intermediate.

The same synthesis method as in Synthesis Example 1 was used, except that raw materials Sub-2 to Sub-6 in the following Table 1 were respectively used in step 4 to replace Sub-1 to prepare different deuterated composition intermediates Sub-A-2 to Sub-A-6.

TABLE 1
Raw materials for synthesis of deuterated composition intermediates

| | | | Average degree |
|---|---|---|---|
| | | | of deuteration |
| | Deuterated composition intermediate | | of deuterated |
| | (with statistical data of degrees of deuteration | Nuclear magnetic | composition |
| Raw material | at sites of deuteration) | resonance analysis | intermediate |
| 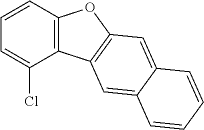 [SMILES: Clc1cccc2oc3cc4ccccc4cc3c12] | 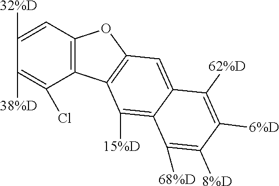 | ¹H NMR (400 MHz,) δ 8.37(1H), 8.02-8.01(0.32H), 8.01-8.00(0.38H), 7.97-7.94(0.85H), 7.93-7.91(0.94H), 7.55-7.51(0.92H), 7.51-7.50(0.68H), 7.48-7.46(0.62H), 7.31-7.27(1H) | 25% |
| 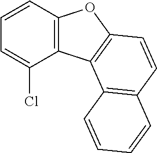 [SMILES: Clc1cccc2oc3ccc4ccccc4c3c12] | 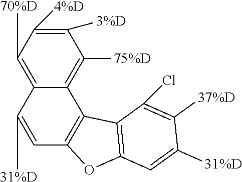 | ¹H NMR (400 MHz) δ 8.56- 8.54(0.97H), 8.30-8.28(1H), 8.04-8.02(0.69H), 7.96-7.94(0.96H), 7.77(0.30H), 7.33 (0.25H), 7.51-7.70 (1H), 7.58-7.55 (0.69H), 7.47-7.45 (0.63H). | 28% |
| 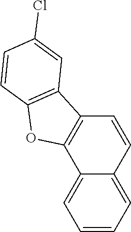 [SMILES: Clc1ccc2oc3c4ccccc4ccc3c2c1] | 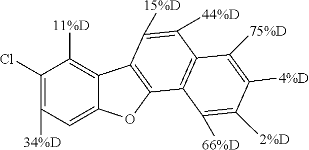 | ¹H NMR (400 MHz) δ 8.48- 8.46(0.34H), 8.05-8.03(0.98H), 8.01-7.99(0.96H), 7.97(0.56H), 7.84-7.82(0.85H), 7.72(0.25H), 7.68-7.65(1H), 7.63-7.61(0.66H), 7.48-7.46(0.89H) | 28% |
| 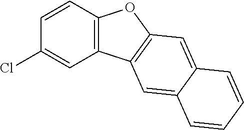 [SMILES: Clc1ccc2oc3cc4ccccc4cc3c2c1] | 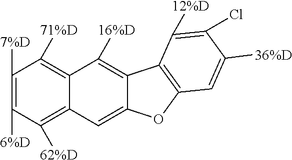 | ¹H NMR (400 MHz) δ 8.41(1H), 8.22-8.21(0.84H), 8.08-8.06(0.94H), 8.02-8.00(0.93H), 7.95(0.88H), 7.66-7.65(0.38H), 7.59-7.55(0.29H), 7.53-7.51(0.64H), 7.49-7.47(1H) | 23% |
| 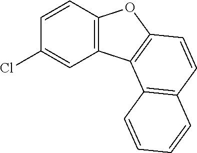 [SMILES: Clc1ccc2oc3ccc4ccccc4c3c2c1] | 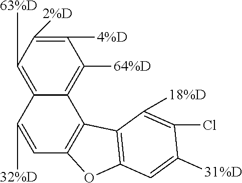 | ¹H NMR (400 MHz) δ 8.58- 8.56(0.96H), 8.39(0.82H), 8.08-8.06(0.68H), 8.01-7.99(0.98H), 7.80-7.78(0.36H), 7.78-7.77(0.37H), 7.65-7.63(1H), 7.63-7.59(0.82H), 7.51-7.48(0.69H) | 24% |

### Synthesis Example 2

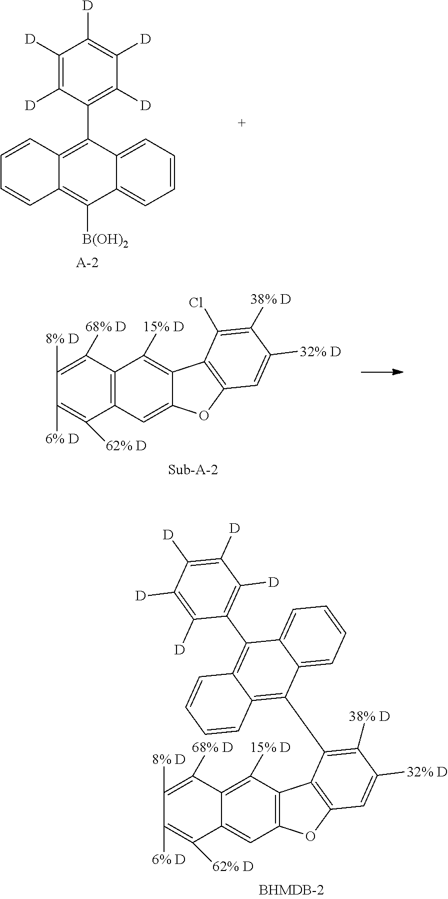

The same method as in Synthesis Example 1 was used, except that Compound A-2 (3.0 g, 10 mmol) and Compound Sub-A-2 (2.8 g) were used as raw materials to prepare BHMDB-2 (1.7 g), with a molecular weight of 476-482. After analysis, the statistical data of the degree of deuteration at each site of deuteration for hydrogen susceptible to deuteration in deuterated composition BHMDB-2 was as shown in the reaction equation.

### Synthesis Example 3

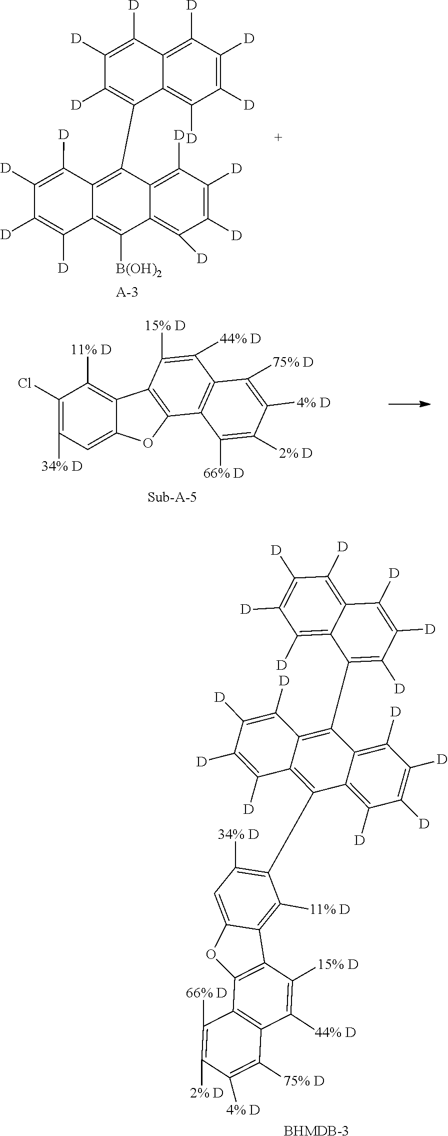

The same method as in Synthesis Example 1 was used, except that Compound A-3 (3.6 g, 10 mmol) and Compound Sub-A-5 (2.8 g) were used as raw materials to prepare BHMDB-3 (3.3 g), with a molecular weight of 536-543. After analysis, the statistical data of the degree of deuteration at each site of deuteration for hydrogen susceptible to deuteration in deuterated composition BHMDB-3 was as shown in the reaction equation.

### Synthesis Example 4

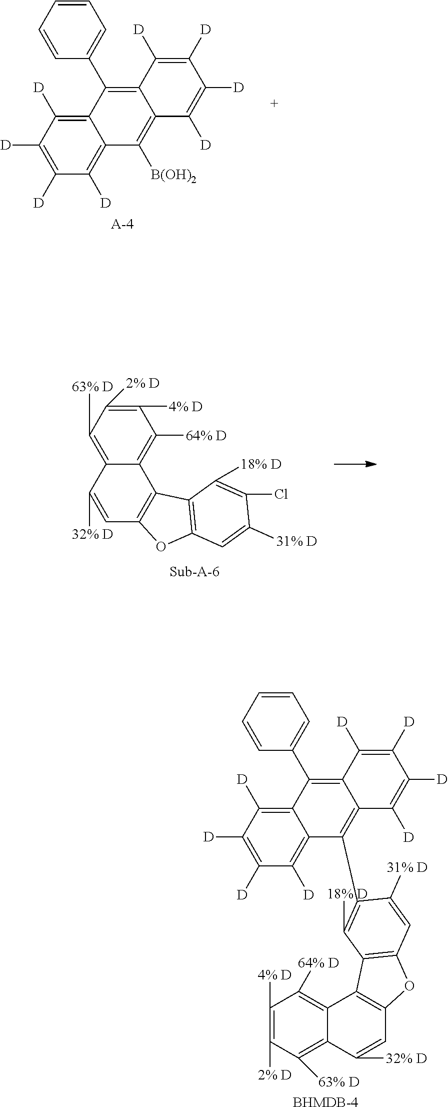

The same method as in Synthesis Example 1 was used, except that Compound A-4 (3.1 g, 10 mmol) and Compound Sub-A-6 (2.8 g) were used as raw materials to prepare BHMDB-4 (3.1 g), with a molecular weight of 479-485. After analysis, the statistical data of the degree of deuteration at each site of deuteration for hydrogen susceptible to deuteration in deuterated composition BHMDB-4 was as shown in the reaction equation.

### Synthesis Example 5

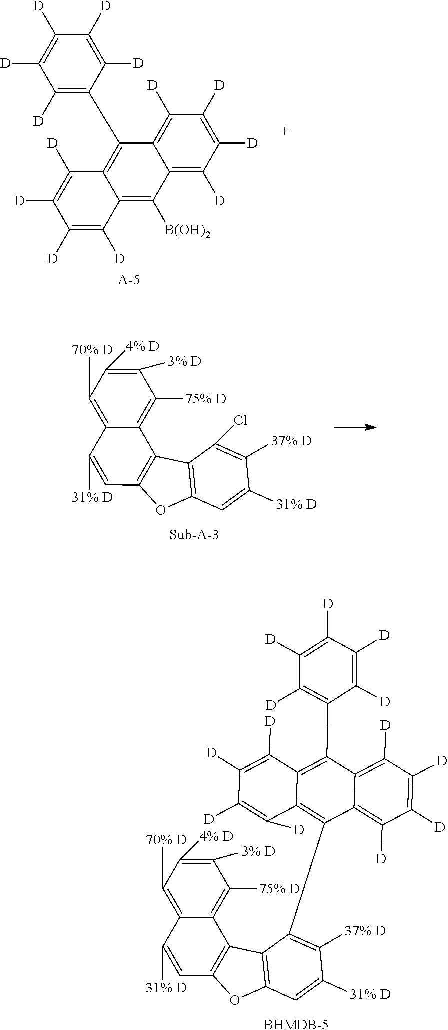

The same method as in Synthesis Example 1 was used, except that Compound A-5 (2.9 g, 10 mmol) and Compound Sub-A-3 (2.8 g) were used as raw materials to prepare BHMDB-5 (3.1 g), with a molecular weight of 484-490. After analysis, the statistical data of the degree of deuteration at each site of deuteration for hydrogen susceptible to deuteration in deuterated composition BHMDB-5 was as shown in the reaction equation.

### Synthesis Example 6

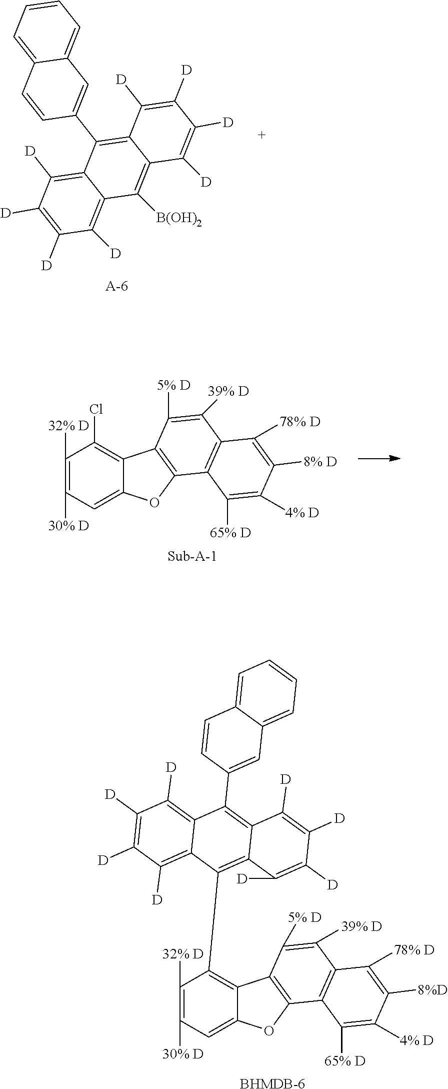

The same method as in Synthesis Example 1 was used, except that Compound A-6 (3.6 g, 10 mmol) and Compound Sub-A-1 (2.8 g) were used as raw materials to prepare BHMDB-6 (3.0 g), with a molecular weight of 529-536. After analysis, the statistical result of the degree of deuteration at each site of deuteration for hydrogen susceptible to deuteration in deuterated composition BHMDB-6 was as shown in the reaction equation.

### Synthesis Example 7

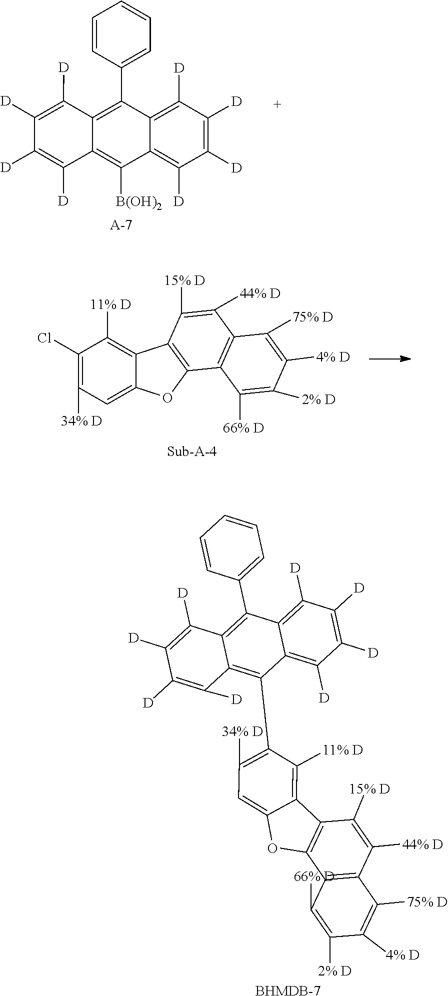

The same method as in Synthesis Example 1 was used, except that Compound A-7 (3.0 g, 10 mmol) and Compound Sub-A-4 (2.8 g) were used as raw materials to prepare BHMDB-7 (2.6 g), with a molecular weight of 479-486. After analysis, the statistical data of the degree of deuteration at each site of deuteration for hydrogen susceptible to deuteration in deuterated composition BHMDB-7 was as shown in the reaction equation.

### Synthesis Example 8

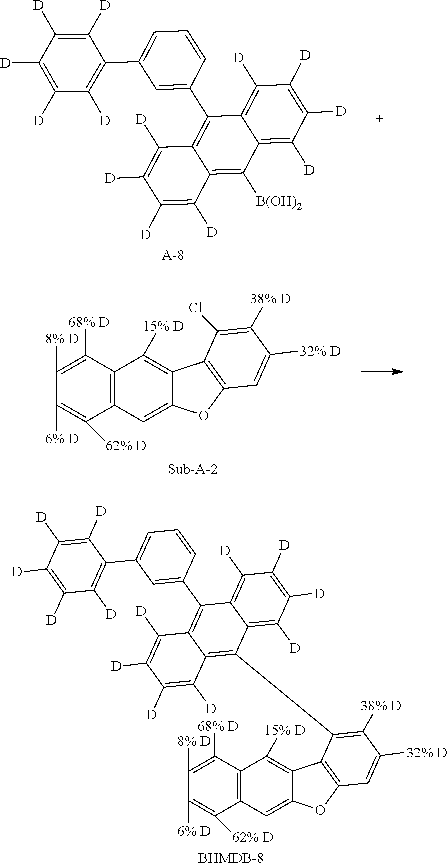

The same method as in Synthesis Example 1 was used, except that Compound A-8 (3.9 g, 10 mmol) and Compound Sub-A-2 (2.8 g) were used as raw materials to prepare BHMDB-8 (1.9 g), with a molecular weight of 560-566. After analysis, the statistical data of the degree of deuteration at each site of deuteration for hydrogen susceptible to deuteration in deuterated composition BHMDB-8 was as shown in the reaction equation.

### Synthesis Example 9

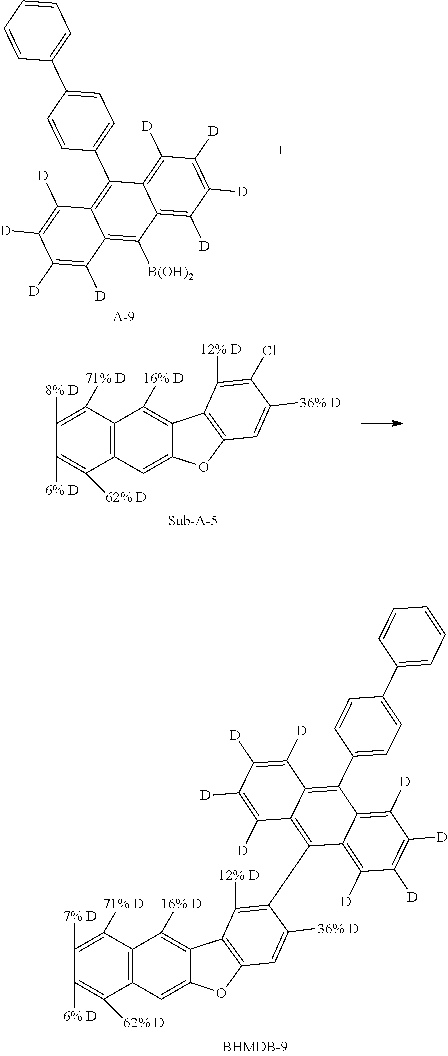

The same method as in Synthesis Example 1 was used, except that Compound A-9 (3.8 g, 10 mmol) and Compound Sub-A-5 (2.8 g) were used as raw materials to prepare BHMDB-9 (1.7 g), with a molecular weight of 555-561. After analysis, the statistical data of the degree of deuteration at each site of deuteration for hydrogen susceptible to deuteration in deuterated composition BHMDB-9 was as shown in the reaction equation.

### Synthesis Example 10

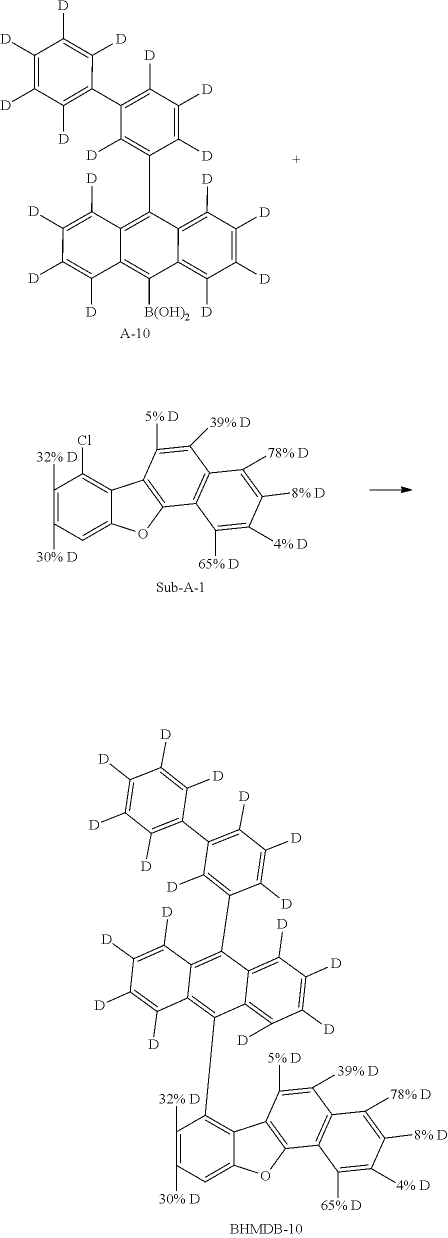

The same method as in Synthesis Example 1 was used, except that Compound A-1 (3.9 g, 10 mmol) and Compound Sub-A-1 (2.8 g) were used as raw materials to prepare BHMDB-10 (2.1 g), with a molecular weight of 464-571. After analysis, the statistical data of the degree of deuteration at each site of deuteration for hydrogen susceptible to deuteration in deuterated composition BHMDB-10 was as shown in the reaction equation.

### Comparative Synthesis Example 1

Preparation of intermediate

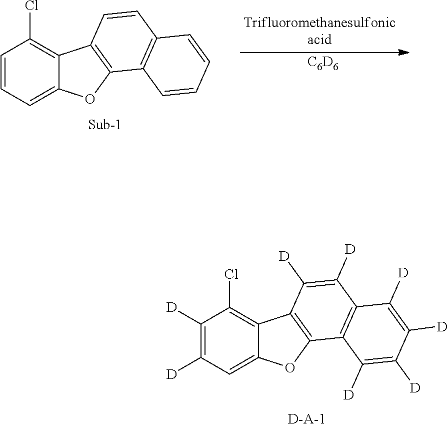

To a 500 ml three-necked flask were added 200 ml of deuterated benzene and Sub-1 (20 g, 79 mmol), followed by the addition of trifluoromethanesulfonic acid (23.7 g, 158 mmol) in portions. The reaction system was then stirred and reacted under reflux for 4 hours. After the reaction was complete, the reaction system was cooled to room temperature, 500 ml of water was added and stirred for 1 hour, during which a solid precipitated out. After suction filtration, the filtrate was subjected to liquid separation to obtain an organic phase. After the organic phase was concentrated, the concentrated organic phase was first separated using a column (dichloromethane:toluene=1:1, v/v) for preliminary purification and then recrystallized five times with a mixed liquid of a mixed liquid of dichloromethane and n-hexane (the volume ratio of dichloromethane to n-hexane being 1:1) to obtain product D-A-1 (8.3 g).

### Preparation of Comparative Compound D12

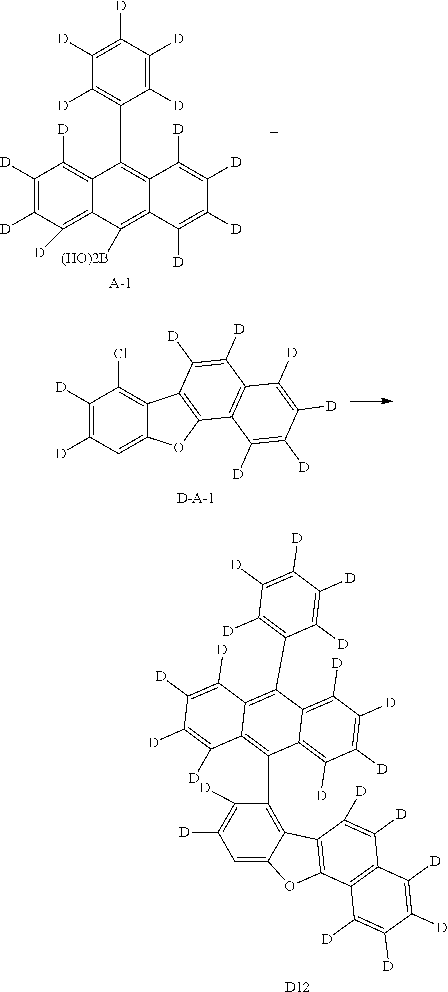

To a 250 ml three-necked flask were added Compound A-1 (3.1 g, 10 mmol), Compound D-A-1 (2.8 g), potassium carbonate (2.1 g, 15 mmol), tetrakis(triphenylphosphine)palladium (0.58 g, 0.5 mmol), and 100 ml of DMF. The mixture was uniformly stirred under nitrogen protection. Subsequently, the reaction system was heated to 100° C. under nitrogen protection and reacted for 4 hours. After the reaction was complete, the reaction system was cooled to room temperature, 500 ml of toluene and 500 ml of water were added, followed by liquid separation. After the organic is phase was concentrated, the concentrated organic phase was purified using a column to obtain 1.8 g of Compound D12, with a molecular weight of 492.

By traditional acid catalysis methods, due to the strong catalytic effect of trifluoromethanesulfonic acid, the obtained product had a fully deuterated structure. The product was further subjected to a coupling reaction to prepare fully deuterated Comparative Compound D12. In addition, due to the relatively high reaction activity and the low product purity, the overall yield of the final product was significantly reduced.

### Comparative Synthesis Example 2

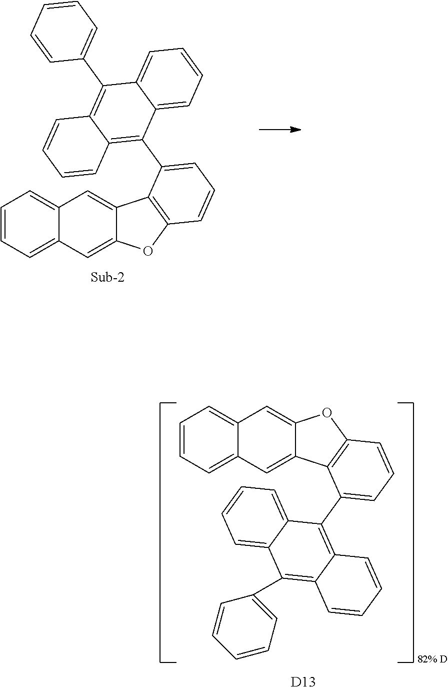

To a 250 ml three-necked flask were added 100 ml of deuterated benzene and Sub-2 (9.4 g, 20 mmol), followed by the addition of trifluoromethanesulfonic acid (6.3 g, 40 mmol) in portions. The reaction system was then stirred and reacted under reflux for 4 hours. After the reaction was complete, the reaction system was cooled to room temperature, 500 ml of water was added and stirred for 1 hour, during which a solid precipitated out. After suction filtration, the filtrate was subjected to liquid separation to obtain an organic phase. After the organic phase was concentrated, the concentrated organic phase was first separated using a column (dichloromethane:toluene=1:1, v/v) for preliminary purification and then recrystallized five times with a mixed liquid of a mixed liquid of dichloromethane and n-hexane (the volume ratio of dichloromethane to n-hexane being 1:1) to obtain Comparative Composition D13 (2.1 g). After calculation by mass spectrometry, the degree of deuteration of Compound D13 was about 82%.

### Comparative Compounds and Compositions

The following lists some of the compounds and compositions that have been studied.

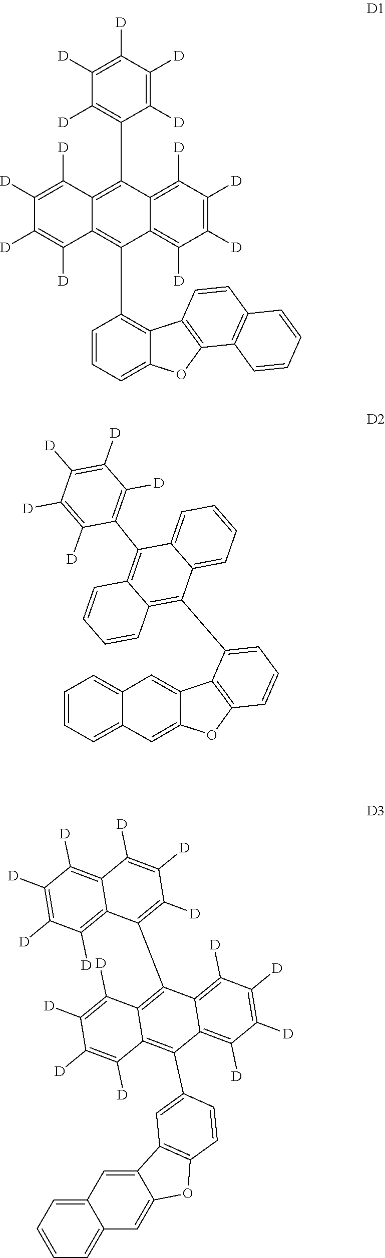 [SMILES: [2H]c1c([2H])c([2H])c(-c2c3c([2H])c([2H])c([2H])c([2H])c3c(-c3cccc4oc5c6ccccc6ccc5c34)c3c([2H])c([2H])c([2H])c([2H])c23)c([2H])c1[2H] ; [2H]c1c([2H])c([2H])c(-c2c3ccccc3c(-c3cccc4oc5cc6ccccc6cc5c34)c3ccccc23)c([2H])c1[2H] ; [2H]c1c([2H])c([2H])c2c(-c3c4c([2H])c([2H])c([2H])c([2H])c4c(-c4ccc5oc6cc7ccccc7cc6c5c4)c4c([2H])c([2H])c([2H])c([2H])c34)c([2H])c([2H])c([2H])c2c1[2H]]

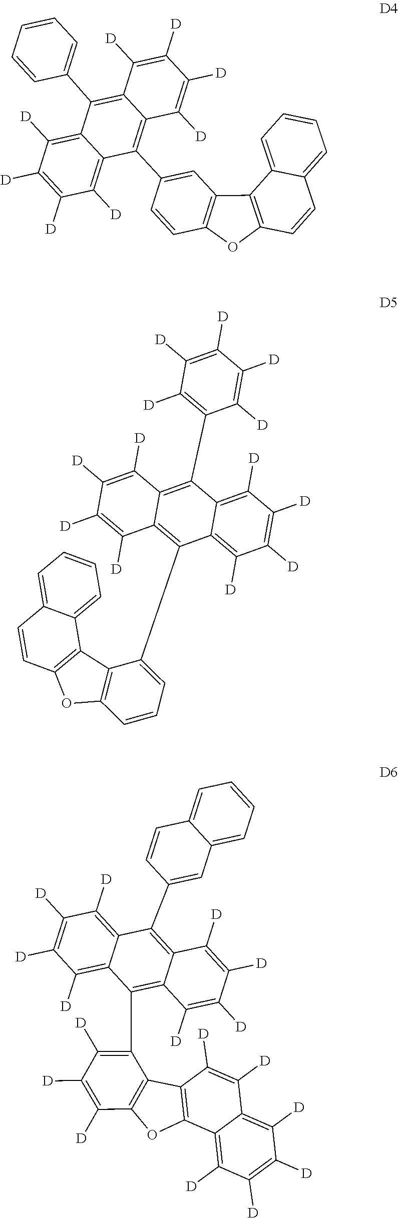 [SMILES: [2H]c1c([2H])c([2H])c2c(-c3ccc4oc5ccc6ccccc6c5c4c3)c3c([2H])c([2H])c([2H])c([2H])c3c(-c3ccccc3)c2c1[2H] ; [2H]c1c([2H])c([2H])c(-c2c3c([2H])c([2H])c([2H])c([2H])c3c(-c3cccc4oc5ccc6ccccc6c5c34)c3c([2H])c([2H])c([2H])c([2H])c23)c([2H])c1[2H] ; [2H]c1c([2H])c(-c2c3c([2H])c([2H])c([2H])c([2H])c3c(-c3ccc4ccccc4c3)c3c([2H])c([2H])c([2H])c([2H])c23)c2c(oc3c4c([2H])c([2H])c([2H])c([2H])c4c([2H])c([2H])c32)c1[2H]]

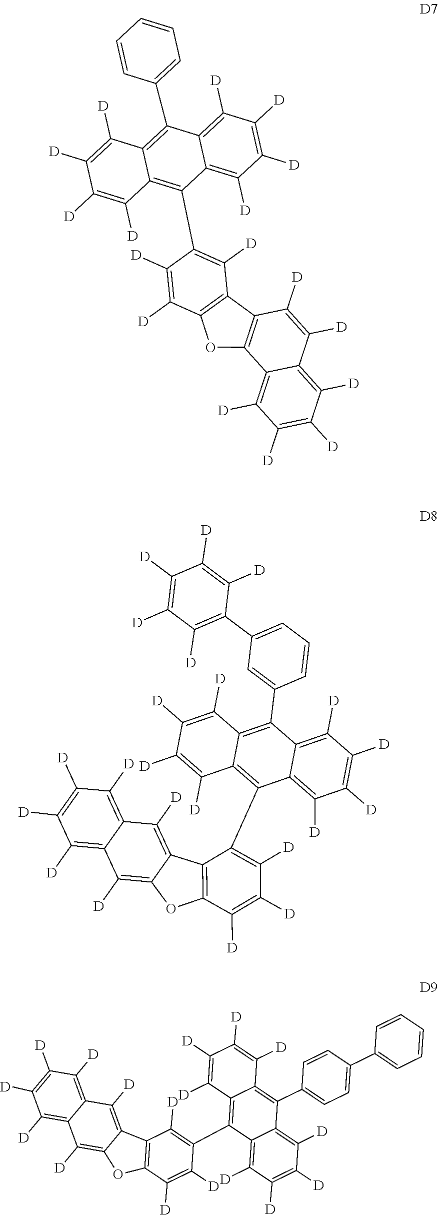

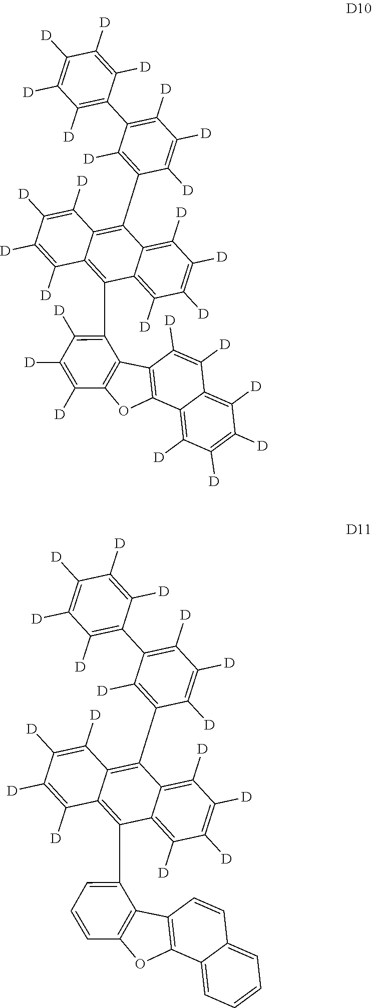 [SMILES: [2H]c1c([2H])c([2H])c(-c2c([2H])c([2H])c([2H])c(-c3c4c([2H])c([2H])c([2H])c([2H])c4c(-c4c([2H])c([2H])c([2H])c5oc6c7c([2H])c([2H])c([2H])c([2H])c7c([2H])c([2H])c6c45)c4c([2H])c([2H])c([2H])c([2H])c34)c2[2H])c([2H])c1[2H] ; [2H]c1c([2H])c([2H])c(-c2c([2H])c([2H])c([2H])c(-c3c4c([2H])c([2H])c([2H])c([2H])c4c(-c4cccc5oc6c7ccccc7ccc6c45)c4c([2H])c([2H])c([2H])c([2H])c34)c2[2H])c([2H])c1[2H]]

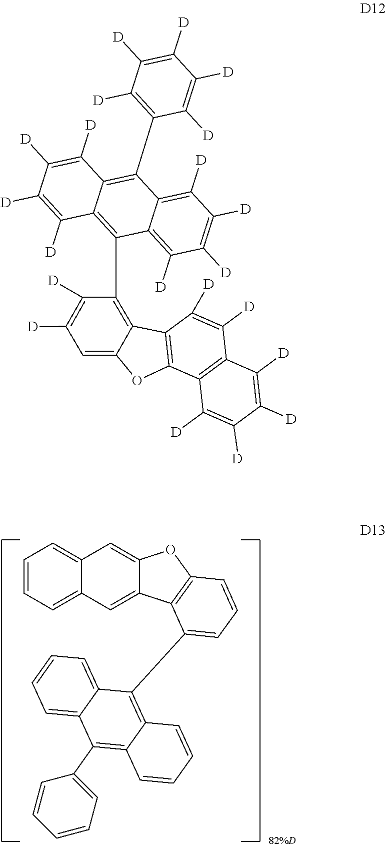

### Properties of Compositions

To better illustrate the characteristics of the deuterated compositions provided by the present invention, the following experiments were conducted.

The compositions BHMDB-1 to BHMDB-10 obtained from the above synthesis examples were used as Examples 1-10 for experiments. The above Comparative Compounds D1-D10 were respectively used as Comparative Examples 1-10 for experiments. D10 and D11 mixed at mass ratios of 1:3, 1:1, and 3:1 were respectively used as Comparative Examples 11-13 for experiments. Comparative Compounds D12 and D13 were respectively used as Comparative Examples 14 and 15 for experiments.

An organic thin film roughness test was carried out, and the specific experimental method was described as follows.

On a quartz glass substrate, the compositions or compounds provided in the above examples and comparative examples were deposited in vacuo at a rate of 1 Å/s to form 50 nm-thick organic thin films as the samples to be tested. Subsequently, the samples to be tested were heated to 150° C. in a nitrogen atmosphere, maintained at that temperature for 30 min, and then returned to room temperature. This operation was repeated three times. Subsequently, the surface roughnesses (R^{a}) of the samples were tested by an atomic force microscope (AFM). The greater the surface roughness, the worse the film-forming property of the surface.

Solution method and thin film PL spectrum tests were carried out, and the specific experimental method was as described below.

The compositions or compounds provided in the above examples and comparative examples were dissolved in toluene to prepare solutions having a concentration of about 1×10⁻⁵ mol/L as the samples to be tested for PL spectra in the solution state.

On a quartz glass substrate, the compositions or compounds provided in the above examples and comparative examples were deposited in vacuo at a rate of 1 Å/s to form 50 nm-thick thin films as the samples to be tested. Subsequently, the samples to be tested were heated to 150° C. in a nitrogen atmosphere, maintained at that temperature for 30 min, and then returned to room temperature. This operation was repeated three times. Subsequently, the PL spectra thereof in the thin film state were tested.

The above test results are as shown in Table 2 below.

TABLE 2

| | | Solution state | Thin film state |
|---|---|---|---|
| | Surface | Fluorescence | Fluorescence |
| Organic | roughness | spectrum peak | spectrum peak |
| thin film | (Ra) | (nm) | (nm) |
| Example 1 | 1.8 | 419 | 429 |
| Example 2 | 1.9 | 423 | 433 |
| Example 3 | 2.2 | 415 | 425 |
| Example 4 | 2.1 | 413 | 423 |
| Example 5 | 2.7 | 420 | 429 |
| Example 6 | 2.5 | 415 | 424 |
| Example 7 | 2.2 | 413 | 422 |
| Example 8 | 2.6 | 421 | 430 |
| Example 9 | 2.7 | 414 | 423 |
| Example 10 | 2.4 | 424 | 434 |
| Comparative | 4.1 | 419 | 432 |
| Example 1 | | | |
| Comparative | 4.5 | 423 | 438 |
| Example 2 | | | |
| Comparative | 4.8 | 415 | 430 |
| Example 3 | | | |
| Comparative | 4.3 | 413 | 426 |
| Example 4 | | | |
| Comparative | 4.8 | 420 | 433 |
| Example 5 | | | |
| Comparative | 4.0 | 415 | 430 |
| Example 6 | | | |
| Comparative | 4.5 | 413 | 427 |
| Example 7 | | | |
| Comparative | 5.3 | 421 | 435 |
| Example 8 | | | |
| Comparative | 5.2 | 414 | 427 |
| Example 9 | | | |
| Comparative | 4.3 | 424 | 437 |
| Example 10 | | | |
| Comparative | 4.1 | 424 | 437 |
| Example 11 | | | |
| Comparative | 4.2 | 424 | 437 |
| Example 12 | | | |
| Comparative | 4.2 | 424 | 437 |
| Example 13 | | | |
| Comparative | 4.1 | 419 | 432 |
| Example 14 | | | |
| Comparative | 4.3 | 423 | 437 |
| Example 15 | | | |

As can be seen from Table 2, by measuring the roughness parameter (R^{a}) of the organic thin films, it was found that the organic thin films formed by the deuterated compositions provided by the present invention still had relatively flat surfaces after frequent temperature changes and exhibited excellent thermal stability, which was beneficial to improving the crystallization problem. Moreover, compared with the comparative examples, the deuterated compositions provided by the present invention had smaller spectral red shifts.

### Manufacturing and Characterization of OLEDs

### DEVICE EXAMPLES

The organic electroluminescent device provided by the present invention comprised an anode, a hole transport region, a luminescent layer, an electron transport region, and a cathode, which were sequentially arranged on a substrate plate.

Furthermore, the hole transport region comprised a hole injection layer, a hole transport layer, and a luminescent auxiliary layer; and the electron transport region is comprised an electron transport layer and an electron injection layer.

Furthermore, the luminescent layer was composed of a host material and a guest material, wherein the host material of the luminescent layer can be composed of one molecular material or a plurality of molecular materials.

The composition of the present invention can be used for the luminescent layer of the above organic electroluminescent device.

The anode in the example was an anode material commonly used in the art, such as ITO, Ag or a multilayer structure thereof. The hole injection layer was made of a hole injection material commonly used in the art and was doped with F4TCNQ, HATCN, NDP-9, etc. The hole transport layer was made of a hole transport material commonly used in the art. The host material in the luminescent layer was the composition provided by the present invention, and the guest material was a guest material commonly used in the art. The electron transport layer was made of an electron transport material commonly used in the art. For the electron injection layer, electron injection materials commonly used in the art, such as Liq, LiF, and Yb, were used. For the cathode, commonly used materials in the art, such as the metals Al and Ag or metal mixtures (Ag-doped Mg, Ag-doped Ca, etc.), were used.

The electrode preparation method and the deposition method for each functional layer in this example were both conventional methods in the art, such as vacuum thermal evaporation or ink-jet printing. No more detailed repetition will be given here. Only some process details and test methods in the preparation process are supplemented as follows:

### Device Example 1

The substrates used in the present invention were all subjected to the following operations: patterning an ITO substrate such that the luminescent area thereof had a size of 3 mm×3 mm, then carrying out an ultrasonic treatment with water/isopropanol, respectively, followed by UV/ozone irradiation and then drying at 100° C., then mounting the ITO substrate on a substrate support in a vacuum deposition device, and adjusting is the pressure such that the vacuum level became 1×10⁻⁷ torr. Subsequently, the following operations were carried out. Firstly, on an ITO layer (anode) formed on the substrate, Compound HTL and Compound P-dopant (at a mass ratio of Compound HTL to Compound P-dopant of 97:3) were deposited in vacuo to a thickness of 10 nm to form a hole injection layer; secondly, on the above hole injection layer, Compound HTL was deposited in vacuo to a thickness of 120 nm to form a hole transport layer; thirdly, on the above hole transport layer, Compound BPrime was deposited in vacuo to a thickness of 5 nm to form a luminescent auxiliary layer; fourthly, on the above luminescent auxiliary layer, a mixture of BHMDB-1 in Synthesis Example 1 of the present invention and Compound BD1 was deposited in vacuo to a thickness of 20 nm to form a luminescent layer, wherein BHMDB-1 was used as a host material and Compound BD-1 as a guest material, and the mass ratio of the host material to the guest material was 98:2; next, on the above luminescent layer, Compound HBL was deposited in vacuo to a thickness of 5 nm to form a hole barrier layer; then, on the above hole barrier layer, Compound ETL and Compound Liq (at a mass ratio of Compound ETL to Compound Liq of 1:1) was deposited in vacuo to a thickness of 30 nm to form an electron transport layer; then, on the above electron transport layer, Yb was deposited in vacuo to a thickness of 1 nm to form an electron injection layer; then, on the above electron injection layer, Mg and Ag (a mass ratio of Mg to Ag of 1:9) were deposited to a thickness of 15 nm to form a cathode; then, on the above cathode, Compound CPL was deposited to a thickness of 50 nm to form a cover layer; and finally, the evaporated substrate was packaged and the cleaned cover plate was subjected to a coating process using a UV adhesive by a coating apparatus. The coated cover plate was then moved to a pressure bonding work stage. The evaporated substrate was placed on top of the cover plate. Finally, the substrate and the cover plate were laminated under the action of a lamination apparatus while the UV adhesive was simultaneously photocured, thereby manufacturing a top-emitting organic electroluminescent device. The structure of the device is shown in FIG. 1.

The molecular structural formulas of the materials in the remaining layers other is than the guest material in the luminescent layer were as follows:

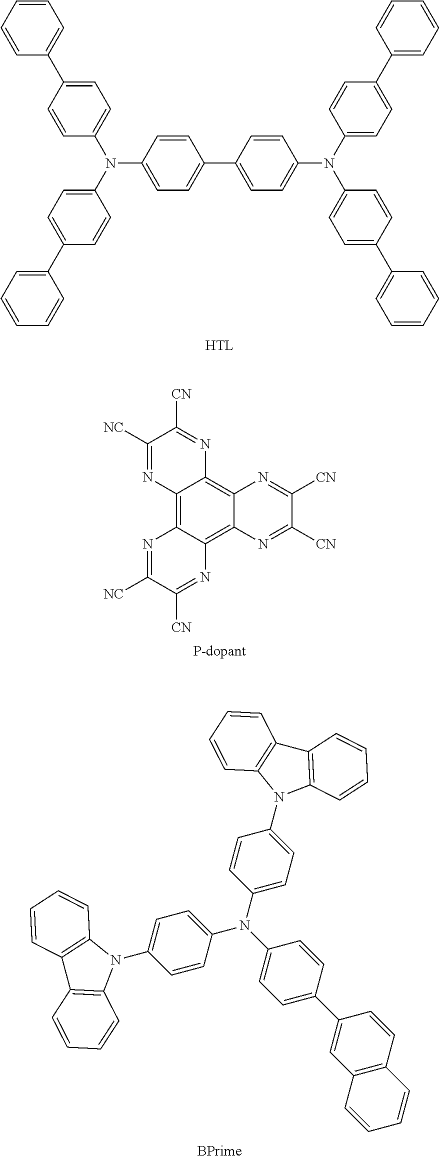 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; N#Cc1nc2c3nc(C#N)c(C#N)nc3c3nc(C#N)c(C#N)nc3c2nc1C#N ; c1ccc2cc(-c3ccc(N(c4ccc(-n5c6ccccc6c6ccccc65)cc4)c4ccc(-n5c6ccccc6c6ccccc65)cc4)cc3)ccc2c1]

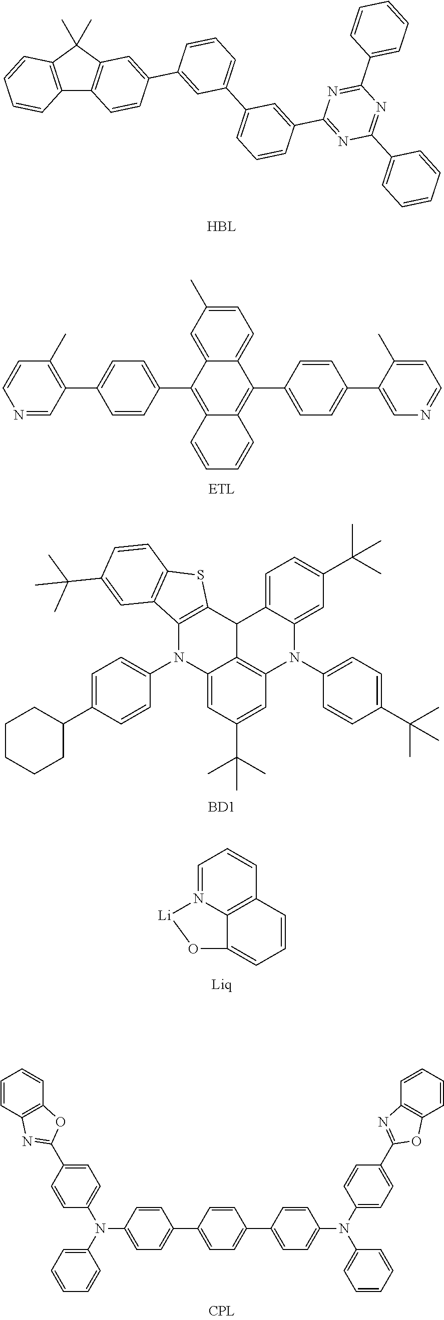

### Device Examples 2-10

Using the above method, the deuterated compositions described in the other examples in Table 2 were prepared into organic electroluminescent devices, which were specifically designated as Device Examples 2-10 of blue-light organic electroluminescent devices, which were prepared respectively by using the deuterated compositions used in the examples in Table 2 instead of the BHMDB-1 in Device Example 1.

### Comparative Device Examples 1-10, 14, and 15

Using the above method, the compounds and compositions described in Comparative Examples 1-10, 14, and 15 in Table 2 were prepared into organic electroluminescent devices, which were specifically designated as Comparative Device Examples 1-10, 14, and 15 of blue-light organic electroluminescent devices, which were prepared respectively by using the compounds and compositions used in Comparative Examples 1-10, 14, and 15 in Table 2 instead of BHMDB-1 in Device Example 1.

### Comparative Device Examples 11-13

Using the above method, the deuterated compositions described in Comparative Examples 11-13 in Table 2 were prepared into organic electroluminescent devices, which were specifically designated as Comparative Device Examples 11-13 of blue-light organic electroluminescent devices, which were prepared respectively by using the compositions used in Comparative Examples 11-13 in Table 2 instead of Synthesis Example 1 in Device Example 1.

Before a performance test, the prepared devices to be tested should be first heated to 150° C. in a nitrogen environment for 30 min and then returned to room temperature. After the operation was repeated three times, the above OLED devices were then tested by a standard method, which can better reflect the device performance of the devices to be tested after frequent temperature changes. To this end, the driving voltage and is luminous efficiency of the organic electroluminescent devices were determined at a current density of J=10 mA/cm². LT97 means that when the prepared blue-light device operates at J=20 mA/cm², the luminous brightness decreases to 97% of its initial value L₀.

The test instruments and methods for testing the performance of the OLED devices of the above examples and comparative examples were as follows:
the brightness was tested by spectrum scanner PhotoResearch PR-735;
the luminous efficiency C.E (cd/A) and color coordinates (CIEy) were tested by spectrum scanner PhotoResearch PR-735;
the current density and turn-on voltage were tested by digital SourceMeter Keithley 2400;
the luminous efficiency of the blue-light device was greatly influenced by chromaticity, and for blue-light devices, the BI value was generally used in the industry as a basis for the efficiency of blue-light devices, wherein BI (Blue index) was obtained by dividing the luminous efficiency C.E (cd/A) by the color coordinates (CIEy); and
the lifetime test was carried out by using a silicon photoelectric OLED device lifetime test system.

The test results are as shown in Table 3.

TABLE 3
Performance test results of blue-light devices

| | | | Device |
|---|---|---|---|
| | Driving | B.I. | lifetime |
| Device | voltage (V) | (cd/A/CIEy) | LT97 (h) |
| Device Example 1 | 3.42 | 261 | 237 |
| Device Example 2 | 3.49 | 263 | 231 |
| Device Example 3 | 3.49 | 258 | 242 |
| Device Example 4 | 3.46 | 257 | 239 |
| Device Example 5 | 3.43 | 256 | 242 |
| Device Example 6 | 3.41 | 256 | 230 |
| Device Example 7 | 3.41 | 255 | 234 |
| Device Example 8 | 3.47 | 258 | 238 |
| Device Example 9 | 3.48 | 258 | 227 |
| Device Example 10 | 3.43 | 255 | 247 |
| Comparative Device Example 1 | 3.42 | 247 | 213 |
| Comparative Device Example 2 | 3.49 | 249 | 193 |
| Comparative Device Example 3 | 3.49 | 243 | 205 |
| Comparative Device Example 4 | 3.46 | 243 | 200 |
| Comparative Device Example 5 | 3.43 | 241 | 203 |
| Comparative Device Example 6 | 3.41 | 242 | 207 |
| Comparative Device Example 7 | 3.41 | 241 | 200 |
| Comparative Device Example 8 | 3.47 | 244 | 203 |
| Comparative Device Example 9 | 3.48 | 244 | 202 |
| Comparative Device Example 10 | 3.43 | 240 | 202 |
| Comparative Device Example 11 | 3.42 | 243 | 212 |
| Comparative Device Example 12 | 3.42 | 244 | 210 |
| Comparative Device Example 13 | 3.44 | 242 | 208 |
| Comparative Device Example 14 | 3.42 | 247 | 213 |
| Comparative Device Example 15 | 3.49 | 244 | 187 |

As can be seen from the test results provided by Device Examples 1-10 and Comparative Device Examples 1-15, the organic electrolumine scent devices containing the deuterated compositions of the present invention all exhibited significant advantages in driving voltage, luminous efficiency, and lifetime.

Apparently, the above examples of the present invention are only examples provided to clearly illustrate the present invention, rather than limitations on the embodiments of the present invention. For those of ordinary skill in the art, other variations or changes in various forms can also be made on the basis of the above description. It is impossible to enumerate all embodiments here. Any obvious variations or changes arising from the technical solutions of the present invention still fall within the scope of protection of the present invention.

1. A deuterated composition, characterized by comprising at least two compounds represented by Formula I or at least two compounds represented by Formula II; 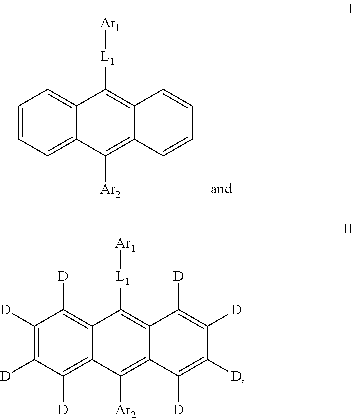
wherein
L₁ represents a single bond or phenylene;
Ar₁ represents deuterated or undeuterated phenyl or naphthyl;
Ar₂ represents one of the following structures: 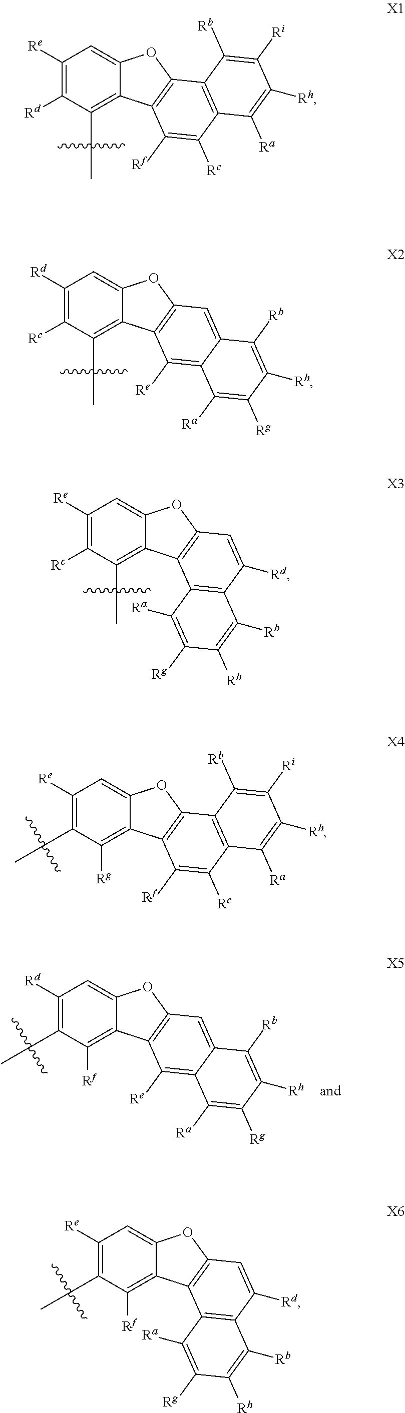
R^{a}, R^{b}, R^{c}, R^{d}, R^{e}, R^{f}, R^{g}, R^{h}, and Rⁱ are the same or different, each independently represent H or D, and satisfy the following conditions:
1) at least one of R^{a} and R^{b} is D; and
2) the degree of deuteration of Ar₂ is 10-60%;
characterized in that Ar₁ represents one of the following structures: 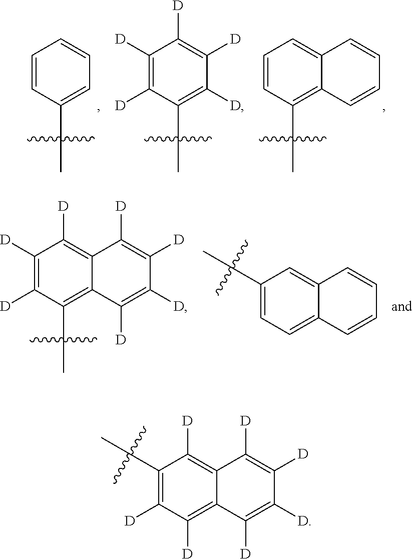
characterized in that in X1 and X4, the degrees of deuteration of R^{a} and R^{b} are both 60%, the degrees of deuteration of R^{c}, R^{d}, and R^{e} are all more than 30%, the degrees of deuteration of R^{f} and R^{g} are both less than 20%, and the degrees of deuteration of R^{h} and Rⁱ are both less than 10%,
characterized in that in X2 and X5, the degrees of deuteration of R^{a} and R^{b} are both more than 60%, the degrees of deuteration of R^{c} and R^{d} are both more than 30%, the degrees of deuteration of R^{e} and R^{f} are both less than 20%, and the degrees of deuteration of R^{g} and R^{h} are both less than 10%,
characterized in that in X3 and X6, the degrees of deuteration of R^{a} and R^{b} are both more than 60%, the degrees of deuteration of R^{c}, R^{d}, and R^{e} are all more than 30%, the degree of deuteration of R^{f} is less than 20%, and the degrees of deuteration of R^{g} and R^{h} are both less than 10%, and
characterized in that in X1 to X6, the degree of deuteration of R^{a} or R^{b} is more than the degree of deuteration of R^{c}, R^{d}, or R^{e}, and the degree of deuteration of R^{c}, R^{d}, or R^{e} is more than the degree of deuteration of R^{f}, R^{g}, R^{h}, or Rⁱ.

2. The deuterated composition according to claim 1, characterized in that at least one of R^{c}, R^{d}, and R^{e} is D; and/or
at least one of R^{f}, R^{g}, R^{h}, and Rⁱ is D.

3. The deuterated composition according to claim 1, characterized in that R^{a} and R^{b} are both D, and R^{c}, R^{d}, R^{e}, R^{f}, R^{g}, R^{h}, and Rⁱ are H or D.

4. The deuterated composition according to claim 1, characterized in that R^{a}, R^{b}, and R^{e} are all D, and R^{c}, R^{d}, R^{f}, R^{g}, R^{h}, Rⁱ are H or D.

5. The deuterated composition according to claim 1, characterized in that R^{a}, R^{b}, R^{c}, and R^{d} are all D, and R^{e}, R^{f}, R^{g}, R^{h}, and Rⁱ are H or D.

6. The deuterated composition according to claim 1, characterized in that R^{a}, R^{b}, R^{c}, R^{d}, and R^{e} are all D, and R^{f}, R^{g}, R^{h}, and Rⁱ are H or D.

7. (canceled)

8. (canceled)

9. (canceled)

10. (canceled)

11. (canceled)

12. The deuterated composition according to claim 1, characterized in that Formula I is selected from one of the following structures: 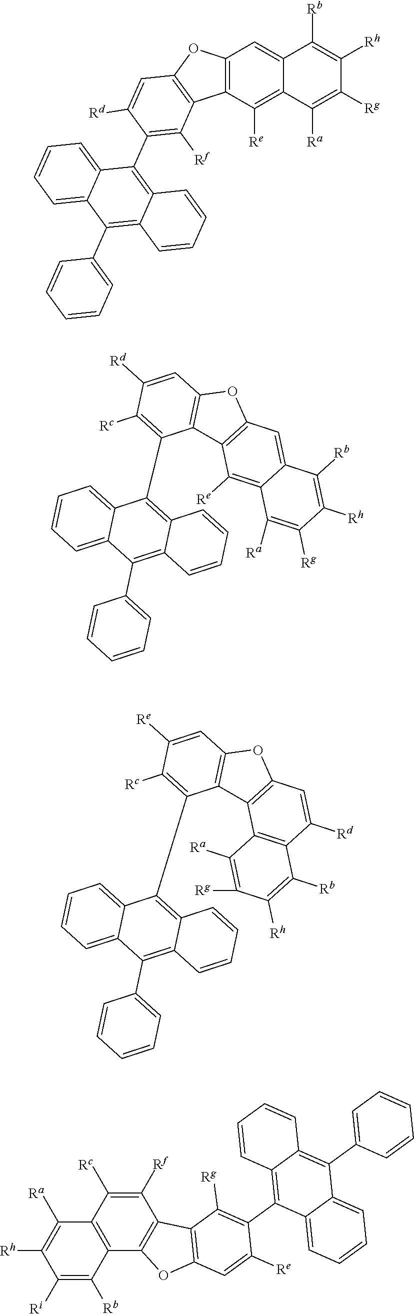 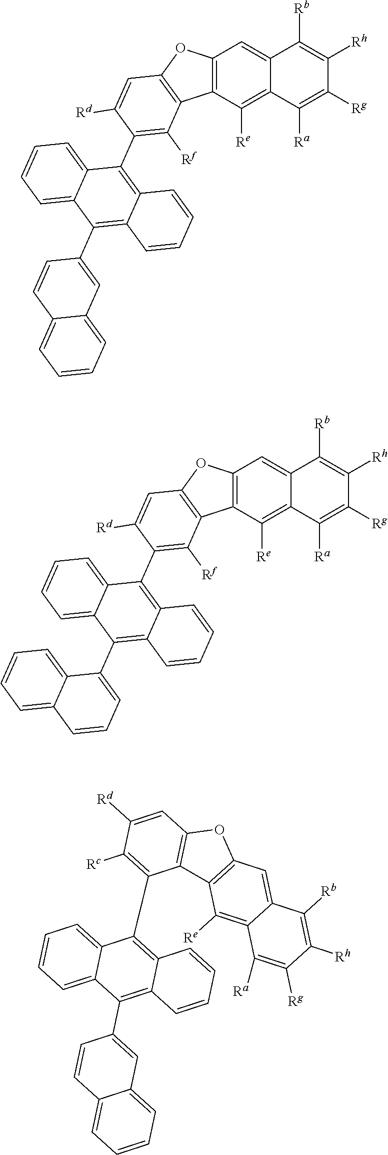 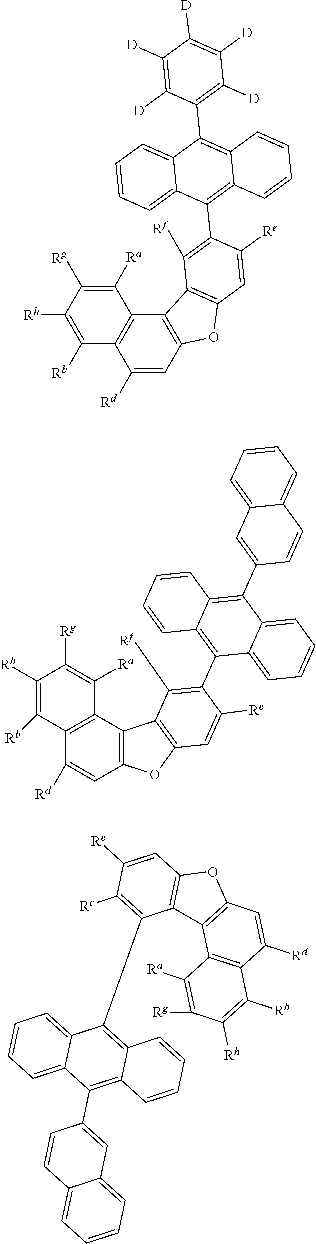 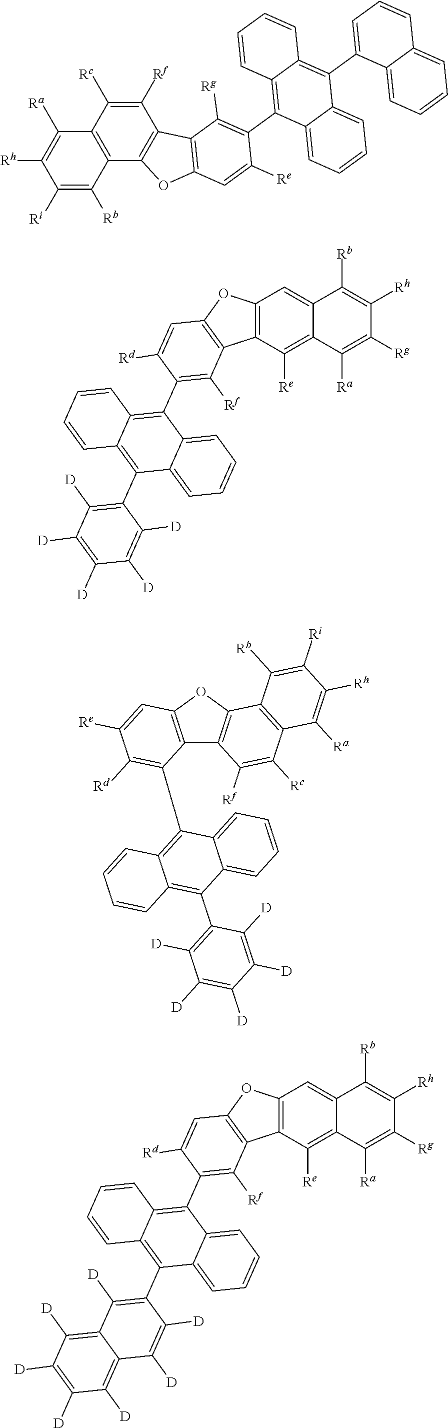 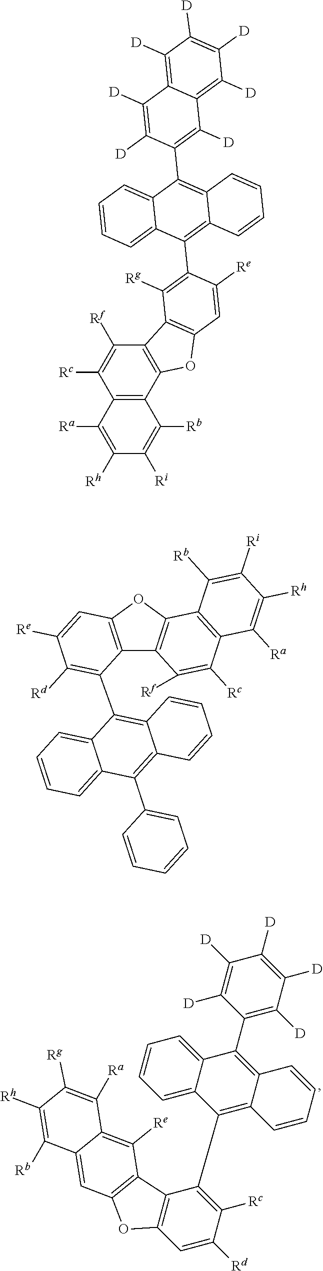
and
Formula II is selected from one of the following structures:

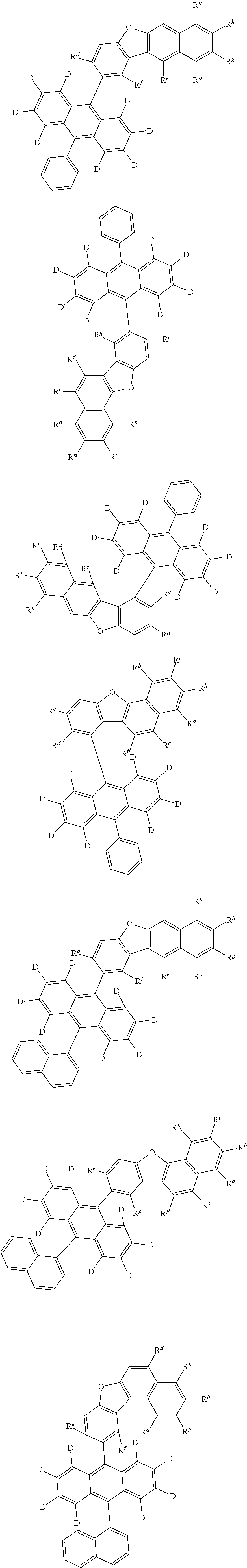

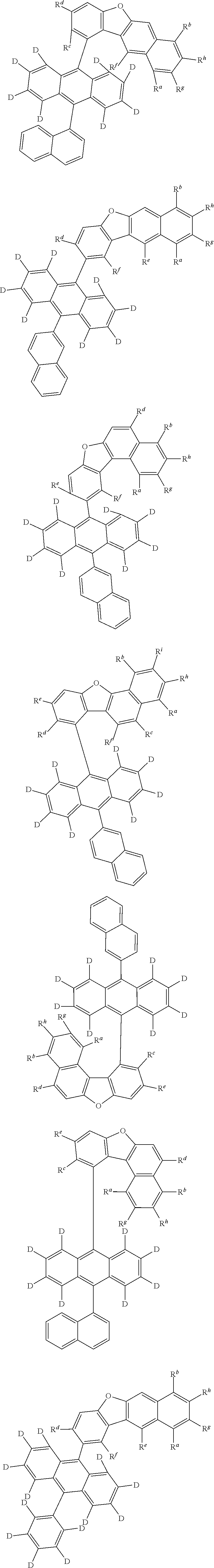

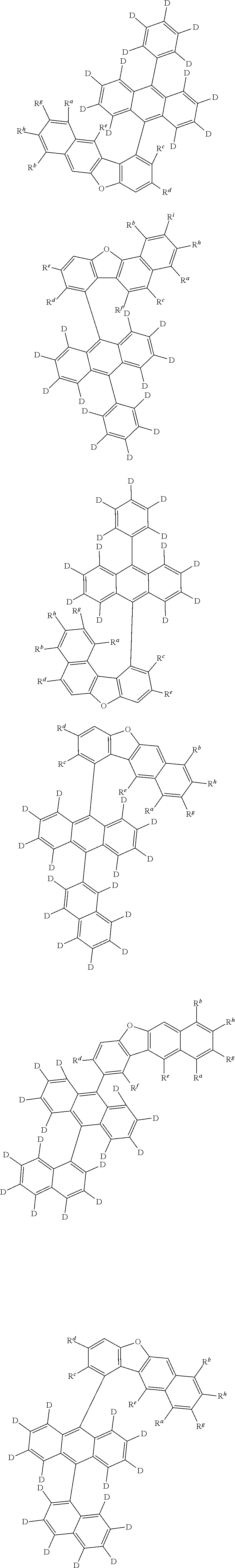

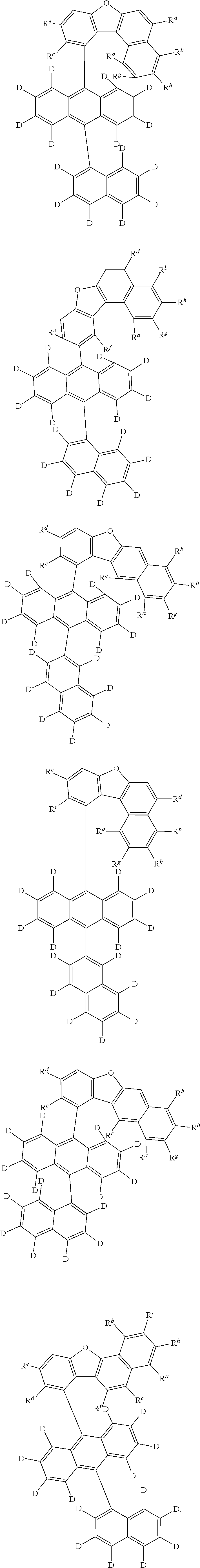

13. A method for preparing the deuterated composition according to claim 12, characterized by comprising the following steps:
S1. adding raw material I, deuterated water, an activated supported catalyst, and an organic solvent to a reaction vessel, and heating and then reacting the mixture to obtain a deuterated composition intermediate, the raw material I being selected from one of structures represented by the following formulas Sub-1 to Sub-6: 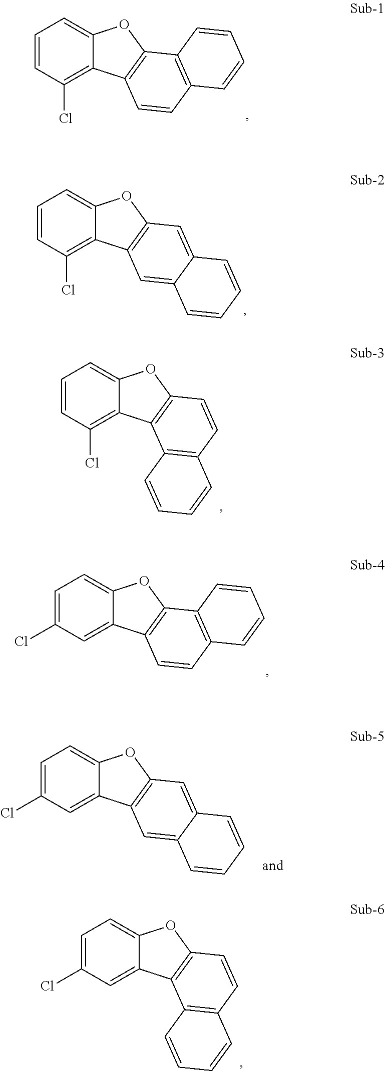 [SMILES: Clc1cccc2oc3c4ccccc4ccc3c12 ; Clc1cccc2oc3cc4ccccc4cc3c12 ; Clc1cccc2oc3ccc4ccccc4c3c12 ; Clc1ccc2oc3c4ccccc4ccc3c2c1 ; Clc1ccc2oc3cc4ccccc4cc3c2c1 ; Clc1ccc2oc3ccc4ccccc4c3c2c1]
wherein the mass ratio of raw material I to deuterated water is 5:1 to 100:1; and
S2. subjecting raw material II, potassium carbonate, a palladium catalyst, and a deuterated composition intermediate to a coupling reaction to obtain the deuterated composition, the raw material II being selected from one of structures represented by the following formulas A-1 to A-2:

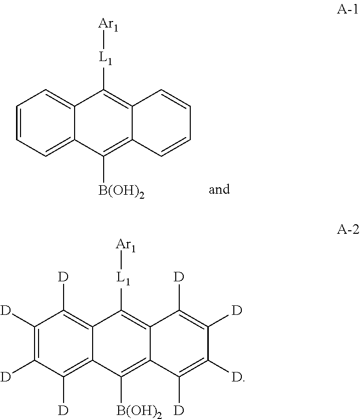

14. The preparation method according to claim 13, characterized in that the supported catalyst is prepared by the following steps:
adding a platinum salt and a palladium salt to dilute hydrochloric acid, stirring and dissolving the platinum salt and palladium salt, then adding a porous material, and subjecting the mixture to impregnation, filtration, drying, and calcination to obtain the supported catalyst,
wherein based on the metal mass, the mass ratio of the porous material to the platinum salt and the palladium salt is 10:0.1 to 10:5.

15. An organic electroluminescent device, characterized by comprising an anode, a hole transport region, a luminescent layer, an electron transport region, and a cathode, which are sequentially arranged on a substrate plate, wherein the luminescent layer comprises a host material and a guest material, and the host material comprises the deuterated composition according to claim 12.
